(19) United States
(12) Patent Application Publication
(10) Pub. No.: US 2005/0271584 A1
(43) Pub. Date: Dec. 8, 2005

(54) Biphenyls and fluorenes as imaging agents in alzheimer's disease

(75) Inventors: Hank F. Kung, Wynnewood, PA (US); Mei Ping Kung, Wynnewood, PA (US); Zhi-Ping Zhuang, Landsdale, PA (US)

Correspondence Address: STERNE, KESSLER, GOLDSTEIN & FOX PLLC, 1100 NEW YORK AVENUE, N.W., WASHINGTON, DC, 20005, US

(73) Assignee: The Trustees of the University of Pennsylvania, Philadelphia, PA (US)

(21) Appl. No.: 10/529,850

(22) PCT Filed: Oct. 6, 2003
(86) PCT No.: PCT/US03/31466

§ 371 Date: Mar. 31, 2005

Related U.S. Application Data
(60) Provisional application No. 60/415,824, filed on Oct. 4, 2002.

(51) Int. Cl. A61K 51/00; C07F 13/00

(52) U.S. Cl. 424/1.11; 534/14

(57) ABSTRACT

This invention relates to a method of imaging amyloid deposits and to labeled compounds, and methods of making labeled compounds useful in imaging amyloid deposits. This invention also relates to compounds, and methods of making compounds for inhibiting the aggregation of amyloid proteins to form amyloid deposits, and a method of delivering a therapeutic agent to amyloid deposits.

### BACKGROUND OF THE INVENTION

1. Field of the Invention

This invention relates to novel bioactive compounds, methods of diagnostic imaging using radiolabeled compounds, and methods of making radiolabeled compounds.

2. Background Art

Alzheimer's disease (AD) is a progressive neurodegenerative disorder characterized by cognitive decline, irreversible memory loss, disorientation, and language impairment. Postmortem examination of AD brain sections reveals abundant senile plaques (SPs) composed of amyloid-β (Aβ) peptides and numerous neurofibrillary tangles (NFTs) formed by filaments of highly phosphorylated tau proteins (for recent reviews and additional citations see Ginsberg, S. D., et al., “Molecular Pathology of Alzheimer's Disease and Related Disorders,” in Cerebral Cortex: Neurodegenerative and Age-related Changes in Structure and Function of Cerebral Cortex, Kluwer Academic/Plenum, NY (1999), pp. 603-654; Vogelsberg-Ragaglia, V., et al., “Cell Biology of Tau and Cytoskeletal Pathology in Alzheimer's Disease,” Alzheimer's Disease, Lippincot, Williams & Wilkins, Philadelphia, Pa. (1999), pp. 359-372). Familial AD (FAD) is caused by multiple mutations in the A precursor protein (APP), presenilin 1 (PS1) and presenilin 2 (PS2) genes (Ginsberg, S. D., et al., “Molecular Pathology of Alzheimer's Disease and Related Disorders,” in Cerebral Cortex: Neurodegenerative and Age-related Changes in Structure and Function of Cerebral Cortex, Kluwer Academic/Plenum, NY (1999), pp. 603-654; Vogelsberg-Ragaglia, V., et al., “Cell Biology of Tau and Cytoskeletal Pathology in Alzheimer's Disease,” Alzheimer's Disease, Lippincot, Williams & Wilkins, Philadelphia, Pa. (1999), pp. 359-372).

While the exact mechanisms underlying AD are not fully understood, all pathogenic FAD mutations studied thus far increase production of the more amyloidogenic 42-43 amino-acid long form of the Aβ peptide. Thus, at least in FAD, dysregulation of Aβ production appears to be sufficient to induce a cascade of events leading to neurodegeneration. Indeed, the amyloid cascade hypothesis suggests that formation of extracellular fibrillar Aβ aggregates in the brain may be a pivotal event in AD pathogenesis (Selkoe, D. J., “Biology of B-amyloid Precursor Protein and the Mechanism of Alzheimer's Disease,” Alzheimer's Disease, Lippincot Williams & Wilkins, Philadelphia, Pa. (1999), pp. 293-310; Selkoe, D. J., J. Am. Med. Assoc. 283:1615-1617 (2000); Naslund, J., et al., J. Am. Med. Assoc. 283:1571-1577 (2000); Golde, T. E., et al., Biochimica et Biophysica Acta 1502:172-187 (2000)).

Various approaches in trying to inhibit the production and reduce the accumulation of fibrillar Aβ in the brain are currently being evaluated as potential therapies for AD (Skovronsky, D. M. and Lee, V. M., Trends Pharmacol. Sci. 21:161-163 (2000); Vassar, R., et al., Science 286:735-741 (1999); Wolfe, M. S., et al., J. Med. Chem. 41:6-9 (1998); Moore, C. L., et al., J Med. Chem. 43:3434-3442 (2000); Findeis, M. A., Biochimica et Biophysica Acta 1502:76-84 (2000); Kuner, P., Bohrmann, et al., J. Biol. Chem. 275:1673-1678 (2000)). It is therefore of great interest to develop ligands that specifically bind fibrillar Aβ aggregates. Since extracellular SPs are accessible targets, these new ligands could be used as in vivo diagnostic tools and as probes to visualize the progressive deposition of Aβ in studies of AD amyloidogenesis in living patients.

To this end, several interesting approaches for developing fibrillar Aβ aggregate-specific ligands have been reported (Ashburn, T. T., et al., Chem. Biol. 3:351-358 (1996); Han, G., et al., J. Am. Chem. Soc. 118:4506-4507 (1996); Klunk, W. E., et al., Biol. Psychiatry 35:627 (1994); Klunk, W. E., et al., Neurobiol. Aging 16:541-548 (1995); Klunk, W. E., et al., Society for Neuroscience Abstract 23:1638 (1997); Mathis, C. A., et al., Proc. XIIth Intl. Symp. Radiopharm. Chem., Uppsala, Sweden:94-95 (1997); Lorenzo, A. and Yankner, B. A., Proc. Natl. Acad. Sci. U.S.A. 91:12243-12247 (1994); Zhen, W., et al., J. Med. Chem. 42:2805-2815 (1999)). One approach is based on highly conjugated chrysamine-G (CG) and Congo red (CR), and the latter has been used for fluorescent staining of SPs and NFTs in postmortem AD brain sections (Ashburn, T. T., et al., Chem. Biol. 3:351-358 (1996); Klunk, W. E., et al., J. Histochem. Cytochem. 37:1273-1281 (1989)). The inhibition constants (Kᵢ) for binding to fibrillar Aβ aggregates of CR, CG, and 3′-bromo- and 3′-iodo derivatives of CG are reported to be 2,800, 370, 300 and 250 nM, respectively (athis, C. A., et al., Proc. XIIth Intl. Symp. Radiopharm. Chem., Uppsala, Sweden:94-95 (1997)). These compounds have been shown to bind selectively to Aβ (1-40) peptide aggregates in vitro as well as to fibrillar Aβ deposits in AD brain sections (athis, C. A., et al., Proc. XIIth Intl. Symp. Radiopharm. Chem., Uppsala, Sweden:94-95 (1997)).

Amyloidosis is a condition characterized by the accumulation of various insoluble, fibrillar proteins in the tissues of a patient. An amyloid deposit is formed by the aggregation of amyloid proteins, followed by the further combination of aggregates and/or amyloid proteins. Formation and accumulation of aggregates of β-amyloid (Aβ) peptides in the brain are critical factors in the development and progression of AD. The fibrillar aggregates of amyloid peptides, Aβ₁₋₄₀ and Aβ₁₋₄₂, are major metabolic peptides derived from amyloid precursor protein found in senile plaques and cerebrovascular amyloid deposits in AD patients (Xia, W., et al., J. Proc. Natl. Acad. Sci. U.S.A. 97:9299-9304 (2000)). Prevention and reversal of Aβ plaque formation are being targeted as a treatment for this disease (Selkoe, D., J. JAMA 283:1615-1617 (2000); Wolfe, M. S., et al., J. Med. Chem. 41:6-9 (1998); Skovronsky, D. M., and Lee, V. M., Trends Pharmacol. Sci. 21:161-163 (2000)).

In addition to the role of amyloid deposits in Alzheimer's disease, the presence of amyloid deposits has been shown in diseases such as Mediterranean fever, Muckle-Wells syndrome, idiopathetic myeloma, amyloid polyneuropathy, amyloid cardiomyopathy, systemic senile amyloidosis, amyloid polyneuropathy, hereditary cerebral hemorrhage with amyloidosis, Down's syndrome, Scrapie, Creutzfeldt-Jacob disease, Kuru, Gerstamnn-Straussler-Scheinker syndrome, medullary carcinoma of the thyroid, Isolated atrial amyloid, β₂-microglobulin amyloid in dialysis patients, inclusion body myositis, β₂-amyloid deposits in muscle wasting disease, and Islets of Langerhans diabetes Type II insulinoma.

Thus, a simple, noninvasive method for detecting and quantitating amyloid deposits in a patient has been eagerly sought. Presently, detection of amyloid deposits involves histological analysis of biopsy or autopsy materials. Both methods have drawbacks. For example, an autopsy can only be used for a postmortem diagnosis.

Imaging agents may be based on two types of isotopes. ^{99m}Tc (T_{1/2}, 6 h; 140 KeV) and ¹²³I (T_{1/2}, 13 h; 159 KeV) are routinely used for single photon emission computed tomography (SPECT), while ¹¹C (T_{1/2}, 20 min; 511 KeV) and ¹⁸F (T_{1/2}, 110 min; 511 KeV) are commonly used for positron emission tomography (PET).

The direct imaging of amyloid deposits in vivo is difficult, as the deposits have many of the same physical properties (e.g., density and water content) as normal tissues. Attempts to image amyloid deposits using magnetic resonance imaging (MRI). and computer-assisted tomography (CAT) have been disappointing and have detected amyloid deposits only under certain favorable conditions. In addition, efforts to label amyloid deposits with antibodies, serum amyloid P protein, or other probe molecules have provided some selectivity on the periphery of tissues, but have provided for poor imaging of tissue interiors.

Potential ligands for detecting Aβ aggregates in the living brain must cross the intact blood-brain barrier. Thus brain uptake can be improved by using ligands with relatively smaller molecular size (compared to Congo Red) and increased lipophilicity. Highly conjugated thioflavins (S and T) are commonly used as dyes for staining the Aβ aggregates in the AD brain (Elhaddaoui, A., et al., Biospectioscopy 1: 351-356 (1995)). These compounds are based on benzothiazole, which is relatively small in molecular size.

It would be useful to have a noninvasive technique for imaging and quantitating amyloid deposits in a patient. In addition, it would be useful to have compounds that inhibit the aggregation of amyloid proteins to form amyloid deposits and a method for determining a compound's ability to inhibit amyloid protein aggregation.

### BRIEF SUMMARY OF THE INVENTION

The present invention provides novel compounds of Formula I, II, III, IV, V or VI.

The present invention also provides diagnostic compositions comprising a radiolabeled compound of Formula I, II, III, IV, V or VI and a pharmaceutically acceptable carrier or diluent.

The invention further provides a method of imaging amyloid depositis, the method comprising introducing into a patient a detectable quantity of a labeled compound of Formula I, II, III, IV, V or VI or a pharmaceutically acceptable salt, ester, amide or prodrug thereof.

The present invention also provides a method for inhibiting the aggregation of amyloid proteins, the method comprising administering to a mammal an amyloid inhibiting amount of a compound Formula I, II, III, IV, V or VI or a pharmaceutically acceptable salt, ester, amide, or prodrug thereof.

A further aspect of this invention is directed to methods and intermediates useful for synthesizing the amyloid inhibiting and imaging compounds of Formula I, II, III, IV, V or VI described herein.

### BRIEF DESCRIPTION OF THE FIGURES

FIG. 1 depicts the structures of potential Aβ plaque imaging agents.

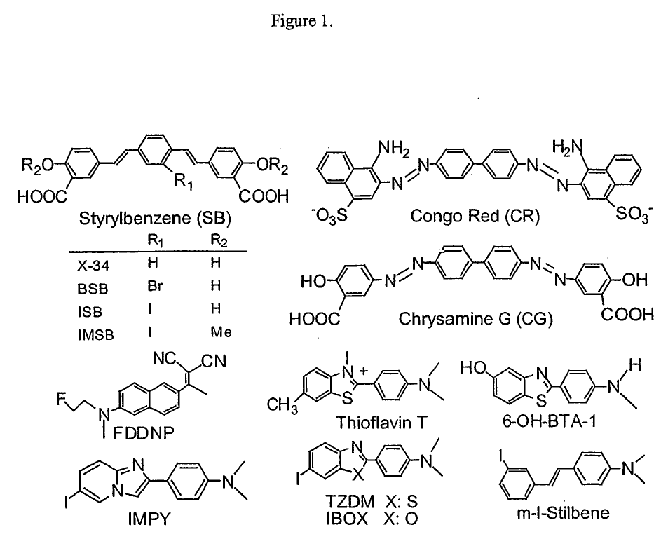

FIG. 2 depicts the binding data for some of the biphenyl and pyrazole compounds of the present invention.

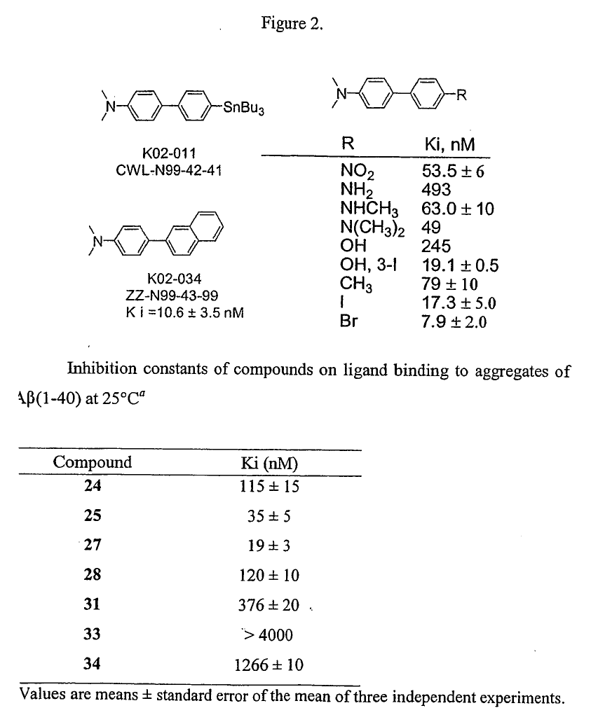

### DETAILED DESCRIPTION OF THE INVENTION

A first aspect of the present invention is directed to compounds of Formula I: 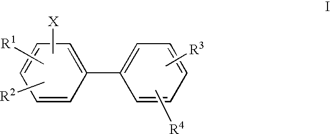
or a pharmaceutically acceptable salt thereof, wherein

R¹, R² and R³ are independently selected from the group consisting of hydrogen, halogen, C₁₋₅ alkyl, cyano, carboxy(C₁₋₅)alkyl, trifluoromethyl, nitro, methylamino, dimethylamino, halo(C₁₋₅)alkyl, hydroxy(C₁₋₅)alkyl, (Bu)₃Sn—, (Bu)₃Sn(C₁₋₅)alkyl, formyl, and the tetradentate metal ligand moiety having the following formula: 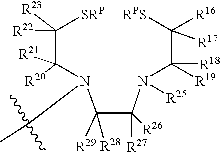
wherein, R¹⁶, R¹⁷, R¹⁸, R¹⁹, R²⁰, R²¹, R²², R²³, R²⁵, R²⁶, R²⁷, R²⁸ and R²⁹ are independently selected from the group consisting of hydrogen, halogen, C₁₋₅ alkyl, cyano, carboxy(C₁₋₅)alkyl, hydroxy(C₁₋₅)alkyl, trifluoromethyl, nitro, methylamino, dimethylamino, halo(C₁₋₅)alkyl, phenyl(C₁₋₅)alkyl, C₃₋₆ cycloalkyl, heterocycle (C₁₋₅)alkyl and carbonyl, and R^{P} is a sulhydryl protecting group,

R⁴ is selected from the group consisting of:
a. C₁₋₅ alkylthio,
b. halo(C₁₋₅)alkyl,
c. halo(C₁₋₅)alkoxy,
d. carboxy(C₁₋₅)alkyl,
e. hydroxy,
f. C₁₋₅ alkoxy,
g. hydroxy(C₁₋₅)alkyl,
h. NR⁵R⁶, wherein
   R⁵ and R⁶ are independently hydrogen, fluoro(C₁₋₅)alkyl or C₁₋₅ alkyl,
i. phenyl(C₁₋₅)alkyl,
j. C₆₋₁₀ aryl,
k. heteroaryl,
l. heterocycle,
m. heterocycle(C₁₋₅)alkyl, and
n. C₃₋₆ cycloalkyl,
   wherein said phenyl(C₁₋₅)alkyl, C₆₋₁₀ aryl, heteroaryl, heterocycle, heterocycle(C₁₋₅)alkyl or C₃₋₆ cycloalkyl is substituted with one of the following: C₁₋₅ alkylthio, C₁₋₅ alkylsulfonyl, methoxy, hydroxy, dimethylamino or methylamino,
      and, X is hydrogen, halogen, ¹²⁵I, ¹²³I, ¹³¹I, ¹⁸F, ⁷⁶Br, ⁷⁷Br or Sn(alkyl)₃.

Useful compounds falling within the scope of Formula I include compounds wherein R¹, R² and R³ are independently selected from the group as described above. Preferably, R¹, R² and R³ are hydrogen, C₁₋₅ alkyl, halo(C₁₋₅)alkyl, (Bu)₃Sn—, (Bu)₃Sn(C₁₋₅)alkyl, or the tetradentate metal ligand moiety described above.

Useful compounds of Formula I also include those compounds wherein R⁴ is as described above. Preferable values of R⁴ under the scope of Formula I include halo(C₁₋₅)alkyl, hydroxy, hydroxy(C₁₋₅)alkyl, C₁₋₅ alkoxy, and NR⁵R⁶, wherein R⁵ and R⁶ are independently hydrogen, halo(C₁₋₅)alkyl or C₁₋₅ alkyl. More preferably, R⁴is NR⁵R⁶, wherein R⁵ and R⁶ are independently hydrogen, halo(C₁₋₅)alkyl or C₁₋₅ alkyl. In a most preferable embodiment, R¹, R² and R³ are hydrogen or the tetradentate metal ligand moiety described above, and R⁴ is NR⁵R⁶, wherein R⁵ and R⁶ are independently hydrogen, halo(C₁₋₅)alkyl or C₁₋₅ alkyl. In this embodiment, it is especially preferred that R¹ and R² are both hydrogen, R³ is the tetradentate metal ligand moiety described above, and R⁴ is NR⁵R⁶, wherein R⁵ and R⁶ are independently hydrogen or methyl. In compounds wherein R³ is the tetradentate metal ligand moiety described above, it is most preferable that X is hydrogen.

Other preferred compounds include those compounds where R¹ is methylamino or dimethylamino, R² is hydrogen, R³ is halo(C₁₋₅)alkyl or (Bu₃)Sn(C₁₋₅)alkyl, R⁴ is hydroxy or hydroxy(C₁₋₅)alkyl, and X is hydrogen. In these embodiments, it is more preferred that R¹and R⁴ are in the para position relative to the bridge, and R³ is in the ortho position relative to R⁴. In more preferred embodiments, R¹ is dimethylamino. More preferred embodiments also include those compounds wherein R³ is fluoro(C₁₋₅)alkyl. Most preferably, R³ is fluoromethyl or fluoroethyl. In preferred embodiments, R⁴ is hydroxy, methoxy or ethoxy. Most preferably, R⁴ is hydroxy.

Some preferred compounds falling under general Formula I have the following structures i-iii: 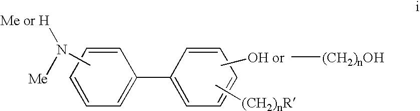
Where, n=1-5 and R′=¹²⁵I, ¹²³I, ¹³¹I, ¹⁸F, ⁷⁶Br, ⁷⁷Br or Sn(alkyl)₃. 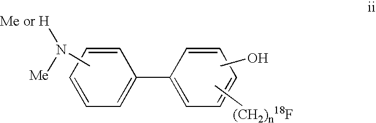
Where, n=1-5.

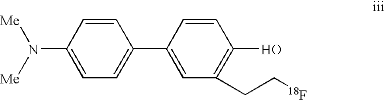

When present in the structure, the tetradentate metal ligand moiety described above is capable of complexing with a metal, such as 99m-pertechnetate, as described herein to from metal chelate derivatives.

Useful values of R¹⁶, R¹⁷, R¹⁸, R¹⁹, R²⁰, R²¹, R²², R²³, R²⁵, R²⁶, R²⁷, R²⁸ and R²⁹ are described above. Preferably, R¹⁶, R¹⁷, R¹⁸, R¹⁹, R²⁰, R²¹, R²², R²³, R²⁵, R²⁶, R²⁷, R²⁸ and R²⁹ are each independently selected from the group consisting of hydrogen, C₁₋₅ alkyl, hydroxy(C₁₋₅)alkyl and carbonyl. Most useful values include hydrogen.

Useful values of X include hydrogen, halogen, ¹²⁵I, ¹²³I, ¹³¹I, ¹⁸F, ⁷⁶Br, ⁷⁷Br or Sn(alkyl)₃. Unless otherwise specified above, it is preferable that X is ¹²³I or ¹⁸F.

Useful values of R^{P} include sulfhydryl protecting such as methoxymethyl, methoxyethoxymethyl, p-methoxybenzyl and benzyl.

With respect to the relative positions of any substituent on an aromatic ring, it is envisioned that R¹, R², R³, R⁴ and X may occur at ortho, meta, or para positions relative to the linkage bond between the aromatic rings. It is also envisioned that in preferred embodiments wherein each aromatic ring has one substituent, the ortho, meta or para position of each substituent is independent of the substituent on the opposite ring. In compounds containing one substituent on each ring it is preferred that each substituent is independently either in a meta or para position relative to said linkage bond. Most preferably, both substituents are in the para position.

The present invention is also directed to compounds of Formula II: 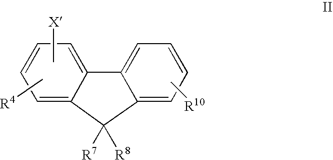
or a pharmaceutically acceptable salt thereof, wherein:

R⁹ and R¹⁰ are independently selected from the group consisting of:
a. hydrogen,
b. C₁₋₅ alkyl,
c. cyano,
d. trifluoromethyl,
e. nitro,
f. halogen,
g. hydroxy(C₁₋₅)alkyl,
h. halo(C₁₋₅)alkyl,
i. C₁₋₅ alkylthio,
j. halo(C₁₋₅)alkoxy,
k. carboxy(C₁₋₅)alkyl,
l. hydroxy,
m. C₁₋₅ alkoxy,
n. NR¹¹R¹², wherein
   R¹¹ and R¹² are independently hydrogen, fluoro(C₁₋₅)alkyl or C₁₋₅ alkyl,
o. phenyl(C₁₋₅)alkyl,
p. C₆₋₁₀ aryl,
q. heteroaryl,
r. heterocycle,
s. heterocycle(C₁₋₅)alkyl, and
t. C₃₋₆ cycloalkyl,
   wherein said phenyl(C₁₋₅)alkyl, C₆₋₁₀ aryl, heteroaryl, heterocycle, heterocycle(C₁₋₅)alkyl or C₃₋₆ cycloalkyl is substituted with one of the following: C₁₋₅ alkylthio, C₁₋₅ alkylsulfonyl, methoxy, hydroxy, dimethylamino or methylamino,
u. the tetradentate metal ligand moiety having the following formula: 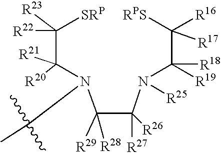
   wherein, R¹⁶, R¹⁷, R¹⁸, R¹⁹, R²⁰, R²¹, R²², R²³, R²⁵, R²⁶, R²⁷, R²⁸ and R²⁹ are independently selected from the group consisting of hydrogen, halogen, C₁₋₅ alkyl, cyano, carboxy(C₁₋₅)alkyl, hydroxy(C₁₋₅)alkyl, trifluoromethyl, nitro, methylamino, dimethylamino, halo(C₁₋₅)alkyl, phenyl(C₁₋₅)alkyl, C₃₋₆ cycloalkyl, heterocycle (C₁₋₅)alkyl and carbonyl, and R^{P} is a sulhydryl protecting group,

R⁷ and R⁸ are independently selected from the group consisting of hydrogen, hydroxy, hydroxy(C₁₋₅)alkyl, C₁₋₅ alkyl, C₁₋₅ alkoxy, halogen, carboxy(C₁₋₅)alkyl, trifluoromethyl, and halo(C₁₋₅)alkyl, phenyl(C₁₋₅)alkyl, C₃₋₆ cycloalkyl, heterocycle(C₁₋₅)alkyl, or R⁷ and R⁸ can be taken together to form a carbonyl,

and, X′ is hydrogen, halogen, ¹²⁵I, ¹²³I, ¹³¹I, ¹⁸F, ⁷⁶Br, ⁷⁷Br or Sn(alkyl)₃.

Useful compounds falling within the scope of Formula II include compounds wherein R⁹ and R¹⁰ are independently selected from the group as described above. Preferably, R⁹ is hydrogen, halogen, hydroxy(C₁₋₅)alkyl, halo(C₁₋₅)alkyl or C₁₋₅ alkyl. Most preferably, R⁹ is hydrogen or the tetradentate metal ligand moiety described above. Preferably, R¹⁰ is selected from the group consisting of cyano, nitro, and NR¹¹R¹², wherein R¹¹ and R¹² are independently hydrogen, halo(C₁₋₅)alkyl or C₁₋₅ alkyl. The most useful value of R¹⁰ is NR¹¹R¹², wherein R¹¹ and R¹² are independently hydrogen or C₁₋₅ alkyl. In this embodiment it is preferred that R¹¹ and R¹² are independently hydrogen, methyl or ethyl. Also preferred are compounds wherein R¹⁰ is NR¹¹R¹² wherein R¹¹ and R¹² are independently hydrogen, methyl or ethyl, X′ is hydrogen, and R⁹ is or the tetradentate metal ligand moiety described above.

Useful compounds are those of Formula II wherein R⁷ and R⁸ are independently selected from the group as described above. Preferably, R⁷ and R⁸ are independently hydrogen, hydroxyl, hydroxy(C₁₋₅)alkyl, halogen, halo(C₁₋₅)alkyl or C₁₋₅ alkyl, or R⁷ and R⁸ are taken together to form a carbonyl. More preferably, R⁷ and R⁸ are independently selected from the group consisting of hydrogen and hydroxyl. In an especially preferred embodiment, R⁷ and R⁸ are both hydrogen.

The tetradentate metal ligand moiety described above is capable of complexing with a metal, such as Tc-99m, as described herein to form metal chelate derivatives (radioisotope complex).

Useful values of R¹⁶, R¹⁷, R¹⁸, R¹⁹, R²⁰, R²¹, R²², R²³, R²⁵, R²⁶, R²⁷, R²⁸ and R²⁹ are described above. Preferably, R¹⁶, R¹⁷, R¹⁸, R¹⁹, R²⁰, R²¹, R²², R²³, R²⁵, R²⁶, R²⁷, R²⁸ and R²⁹ are each independently selected from the group consisting of hydrogen, C₁₋₅ alkyl, and carbonyl. Most useful values include hydrogen.

Useful values of X′ include hydrogen, halogen, ¹²⁵I, ¹²³I, ¹³¹I, ¹⁸F, ⁷⁶Br, ⁷⁷Br or Sn(alkyl)₃. Unless otherwise specified above, it is preferable that X′ is ¹²³I or ¹⁸F.

With respect to the relative positions of any substituent on an aromatic ring, it is envisioned that R⁹, R¹⁰ and X′ may occur at ortho, meta, or para positions relative to the linkage bond between the aromatic rings. It is also envisioned that in preferred embodiments wherein each aromatic ring has one substituent, the ortho, meta or para position of each substituent is independent of the substituent on the opposite ring. In compounds containing one substituent on each ring it is preferred that each substituent is independently either in a meta or para position relative to said linkage bond. Most preferably, both substituents are in the para position.

Another aspect of this invention is directed to compounds of Formula III: 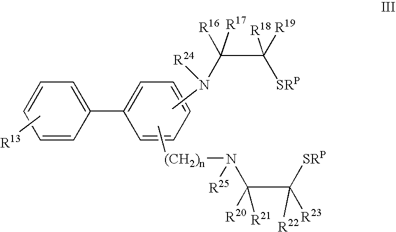
or a pharmaceutically acceptable salt thereof, wherein:

n is zero or one,

R¹³ is selected from the group consisting of:
a. C₁₋₅ alkyl,
b. cyano,
c. trifluoromethyl,
d. nitro,
e. halo(C₁₋₅)alkyl,
f. C₁₋₅ alkylthio,
g. hydroxy(C₁₋₅)alkyl,
h. halogen,
i. halo(C₁₋₅)alkoxy,
j. carboxy(C₁₋₅)alkyl,
k. hydroxy,
l. C₁₋₅ alkoxy,
m. NR¹⁴R¹⁵, wherein
   R¹⁴ and R¹⁵ are independently hydrogen, halo(C₁₋₅)alkyl or C₁₋₅ alkyl,
n. phenyl(C₁₋₅)alkyl,
o. C₆₋₁₀ aryl,
p. heteroaryl,
q. heterocycle,
r. heterocycle(C₁₋₅)alkyl, and
s. C₃₋₆ cycloalkyl,
   wherein said phenyl(C₁₋₅)alkyl, C₆₋₁₀ aryl, heteroaryl, heterocycle, heterocycle(C₁₋₅)alkyl or C₃₋₆ cycloalkyl is substituted with one of the following: C₁₋₅ alkylthio, C₁₋₅ alkylsulfonyl, methoxy, hydroxy, dimethylamino or methylamino,

R¹⁶, R¹⁷, R¹⁸, R¹⁹, R²⁰, R²¹, R²², R²³, R²⁴ and R²⁵ are independently selected from the group consisting of hydrogen, halogen, C₁₋₅ alkyl, cyano, carboxy(C₁₋₅)alkyl, hydroxy(C₁₋₅)alkyl, trifluoromethyl, nitro, methylamino, dimethylamino, halo(C₁₋₅)alkyl, phenyl(C₁₋₅)alkyl, C₃₋₆ cycloalkyl, heterocycle (C₁₋₅)alkyl and carbonyl,

and, R^{P} is a sulfhydryl protecting group such as methoxymethyl, methoxyethoxymethyl, p-methoxybenzyl and benzyl.

Useful compounds falling within the scope of Formula III are those wherein R¹³, R¹⁶, R¹⁷, R¹⁸, R¹⁹, R²⁰, R²¹, R²², R²³, R²⁴ and R²⁵ are as described above. Particularly useful compounds are those wherein R¹³ is NR¹⁴R¹⁵, wherein R¹⁴ and R¹⁵ are independently hydrogen, halo(C₁₋₅)alkyl, hydroxy(C₁₋₅)alkyl or C₁₋₅ alkyl. Preferable values of R¹⁶ and R¹⁷are hydrogen and C₁₋₅ alkyl or R¹⁶ and R¹⁷ are taken together to form a carbonyl. In these preferred compounds, it is more desirable that R¹⁸, R¹⁹, R²⁰, R²¹, R²², R²³, R²⁴ and R²⁵ are independently hydrogen or C₁₋₅ alkyl. In another preferred embodiment, R¹⁶, R¹⁷, R¹⁸, R¹⁹, R²⁰, R²¹, R²², R²³, R²⁴ and R²⁵ are hydrogen.

Useful compounds are those where n equals zero or one. In all embodiments, it is preferred that n equals one.

With respect to the relative positions of any substituent on an aromatic ring, it is envisioned that R¹³ can occur at any available position. The position of R¹³ is independent of the position of any substituent on the opposite ring. In preferred compounds R¹³ is either in a meta or para position relative to the linkage bond between the two aromatic rings. Most preferably, R¹³ is in the para position.

With respect to the metal chelating ligand having two points of attachment to the aromatic ring, it is envisioned that the attachments can occur at any position resulting in a metal chelate ligand capable of complexing a metal atom. Preferably, the attachments are on adjacent atoms of the aromatic ring.

The tetradentate metal ligand moiety of Formula III is capable of complexing with a metal, such as Tc-99m, as described herein to form metal chelate derivatives (radioisotope complex).

Yet another aspect of this invention is directed to a radioisotope complex of Formula III, exemplified by the following Formula: 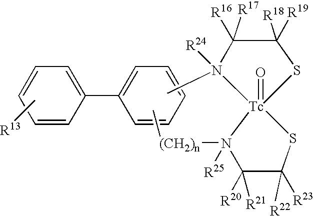
provided that one of R²⁴ and R²⁵ is selected from the group consisting of:
a. hydrogen,
b. C₁₋₅alkyl,
c. trifluoromethyl,
d. halo(C₁₋₅)alkyl,
e. carboxy(C₁₋₅)alkyl,
f. phenyl(C₁₋₅)alkyl,
g. C₆₋₁₀ aryl,
h. heteroaryl,
i. heterocycle,
j. heterocycle(C₁₋₅)alkyl, and
k. C₃₋₆ cycloalkyl,
   wherein said phenyl(C₁₋₅)alkyl, C₆₋₁₀ aryl, heteroaryl, heterocycle, heterocycle(C₁₋₅)alkyl or C₃₋₆ cycloalkyl is substituted with one of the following: C₁₋₅ alkylthio, C₁₋₅ alkylsulfonyl, methoxy, hydroxy, dimethylamino or methylamino,
      the other of R²⁴ and R²⁵ represents an unsubstituted position.

In this aspect of the invention, useful values of R¹³ are as described for Formula III. More useful values of R¹³ are halo(C₁₋₅)alkyl, hydroxy, hydroxy(C₁₋₅)alkyl, C₁₋₅ alkyl, and NR¹⁴R¹⁵, wherein R¹⁴ and R¹⁵ are independently hydrogen, halo(C₁₋₅)alkyl or C₁₋₅ alkyl. More preferably, R¹³ is NR¹⁴R¹⁵, wherein R¹⁴ and R¹⁵ are independently hydrogen, halo(C₁₋₅)alkyl or C₁₋₅ alkyl. In this embodiment, it is preferred that R¹⁸, R¹⁹, R²⁰, R²¹, R²², R²³, R²⁴ and R²⁵ are independently hydrogen or C₁₋₅ alkyl.

Useful compounds are those where n equals zero or one. In all embodiments, it is preferred that n equals one.

With respect to the relative positions of any substituent on an aromatic ring, it is envisioned that R¹³ can occur at any available position. The position of R¹³ is independent of the position of any substituent on the opposite ring.

With respect to the metal chelating ligand having two points of attachment to the aromatic ring, it is envisioned that the attachments can occur at any position resulting in a metal chelate ligand capable of complexing a metal atom. Preferably, the attachments are on adjacent atoms of the aromatic ring.

The present invention is also directed to compounds of Formula IV:
or a pharmaceutically acceptable salt thereof, wherein: 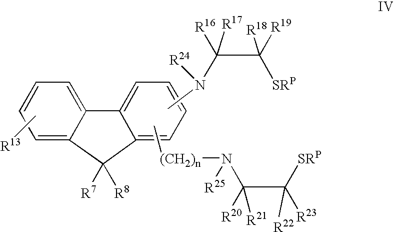
n is zero or one,

R¹³ is selected from the group consisting of:
a. C₁₋₅ alkyl,
b. cyano,
c. trifluoromethyl,
d. nitro,
e. halo(C₁₋₅)alkyl,
f. C₁₋₅ alkylthio,
g. hydroxy(C₁₋₅)alkyl,
h. halogen,
i. halo(C₁₋₅)alkoxy,
j. carboxy(C₁₋₅)alkyl,
k. hydroxy,
l. C₁₋₅ alkoxy,
m. NR¹⁴R¹⁵, wherein
   R¹⁴ and R¹⁵ are independently hydrogen, halo(C₁₋₅)alkyl or C₁₋₅ alkyl,
n. phenyl(C₁₋₅)alkyl,
o. C₆₋₁₀ aryl,
p. heteroaryl,
q. heterocycle,
r. heterocycle(C₁₋₅)alkyl, and
s. C₃₋₆ cycloalkyl,
   wherein said phenyl(C₁₋₅)alkyl, C₆₋₁₀ aryl, heteroaryl, heterocycle, heterocycle(C₁₋₅)alkyl or C₃₋₆ cycloalkyl is substituted with one of the following: C₁₋₅ alkylthio, C₁₋₅ alkylsulfonyl, methoxy, hydroxy, dimethylamino or methylamino,

R¹⁶, R¹⁷, R¹⁸, R¹⁹, R²⁰ R²¹, R²², R²³, R²⁴ and R²⁵ are independently selected from the group consisting of hydrogen, halogen, C₁₋₅ alkyl, cyano, carboxy(C₁₋₅)alkyl, hydroxy(C₁₋₅)alkyl, trifluoromethyl, nitro, methylamino, dimethylamino, halo(C₁₋₅)alkyl, phenyl(C₁₋₅)alkyl, C₃₋₆ cycloalkyl, heterocycle (C₁₋₅)alkyl and carbonyl,

and R^{P} is a sulfhydryl protecting group such as methoxymethyl, methoxyethoxymethyl, p-methoxybenzyl and benzyl.

Useful compounds falling within the scope of Formula IV are those wherein R¹³, R¹⁶, R¹⁷, R¹⁸, R¹⁹, R²⁰, R²¹, R²², R²³, R²⁴ and R²⁵ are as described above. Particularly useful compounds are those wherein R¹³ is hydroxy(C₁₋₅)alkyl or NR¹⁴R¹⁵, wherein R¹⁴ and R¹⁵ are independently hydrogen, halo(C₁₋₅)alkyl or C₁₋₅ alkyl. Preferable values of R¹⁶ and R¹⁷are hydrogen and C₁₋₅ alkyl or R¹⁶ and R¹⁷ are taken together to form a carbonyl. In these preferred compounds, it is more desirable that R¹⁸, R¹⁹, R²⁰, R²¹, R²², R²³, R²⁴ and R²⁵ are independently hydrogen, hydroxy(C₁₋₅)alkyl or C₁₋₅ alkyl. In another preferred embodiment, R¹⁶, R¹⁷, R¹⁸, R¹⁹, R²⁰, R²¹, R²², R²³, R²⁴ and R²⁵ are hydrogen.

Useful compounds are those of Formula IV wherein R⁷ and R⁸ are independently selected from the group as described above. Preferably, R⁷ and R⁸ are independently hydrogen, hydroxyl, hydroxy(C₁₋₅)alkyl, halogen, halo(C₁₋₅)alkyl or C₁₋₅ alkyl, or R⁷ and R⁸ are taken together to form a carbonyl. More preferably, R⁷ and R⁸ are independently selected from the group consisting of hydrogen and hydroxyl. In an especially preferred embodiment, R⁷ and R⁸ are both hydrogen.

Useful compounds are those where n equals zero or one. In all embodiments, it is preferred that n equals one.

With respect to the relative positions of any substituent on an aromatic ring, it is envisioned that R¹³ can occur at any available position. The position of R¹³ is independent of the position of any substituent on the opposite ring. In preferred compounds R¹³ is either in a meta or para position relative to the linkage bond between the two aromatic rings. Most preferably, R¹³ is in the para position.

With respect to the metal chelating ligand having two points of attachment to the aromatic ring, it is envisioned that the attachments can occur at any position resulting in a metal chelate ligand capable of complexing a metal atom. Preferably, the attachments are on adjacent atoms of the aromatic ring. The tetradentate metal ligand moiety of Formula IV is capable of complexing with a metal, such as Tc-99m, as described herein to form metal chelate derivatives (radioisotope complex).

Another aspect of this invention is a radioisotope complex of Formula IV having the following structure: 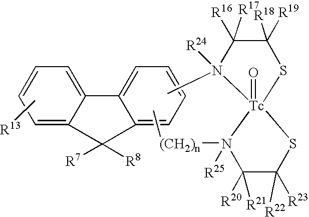
wherein, R¹³, R⁷, and R⁸ are as described for Formula II, and R²⁴ and R²⁵ are as described for the radioisotope complex of Formula III.

The present invention is also directed to compounds of Formula V: 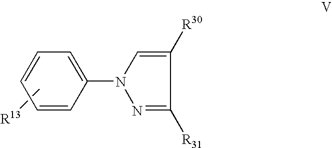
or a pharmaceutically acceptable salt thereof, wherein:

R¹³ is selected from the group consisting of:
a. C₁₋₅ alkyl,
b. cyano,
c. trifluoromethyl,
d. nitro,
e. halo(C₁₋₅)alkyl,
f. C₁₋₅ alkylthio,
g. hydroxy(C₁₋₅)alkyl,
h. halogen,
i. halo(C₁₋₅)alkoxy,
j. carboxy(C₁₋₅)alkyl,
k. hydroxy,
l. C₁₋₅ alkoxy,
m. NR¹⁴R¹⁵, wherein
   R¹⁴ and R¹⁵ are independently hydrogen, halo(C₁₋₅)alkyl or C₁₋₅ alkyl,
n. phenyl(C₁₋₅)alkyl,
o. C₆₋₁₀ aryl,
p. heteroaryl,
q. heterocycle,
r. heterocycle(C₁₋₅)alkyl, and
s. C₃₋₆ cycloalkyl,
   wherein said phenyl(C₁₋₅)alkyl, C₆₋₁₀ aryl, heteroaryl, heterocycle, heterocycle(C₁₋₅)alkyl or C₃₋₆ cycloalkyl is substituted with one of the following: C₁₋₅ alkylthio, C₁₋₅ alkylsulfonyl, methoxy, hydroxy, dimethylamino or methylamino,
      and,

R³⁰ and R³¹ are selected from the group consisting of hydrogen, hydroxy, hydroxy(C₁₋₅)alkyl, C₁₋₅ alkyl, C₁₋₅ alkoxy, (C₁₋₅)alkyl carboxy, halogen, carboxy(C₁₋₅)alkyl, trifluoromethyl, halo(C₁₋₅)alkyl, phenyl(C₁₋₅)alkyl, C₃₋₆ cycloalkyl and heterocycle(C₁₋₅)alkyl, provided,

if R¹³ is other than NR¹⁴R¹⁵, wherein one of R¹⁴ and R¹⁵ is “Fluoro(C₁₋₅)alkyl, then one of R³⁰ and R³¹ is selected from the group consisting of ¹²⁵I, ¹²³I, ¹³¹I, ¹⁸F, ⁷⁶Br, ⁷⁷Br and ¹⁸Fluoro(C₁₋₅)alkyl.

Useful compounds of Formula V are those compounds wherein R¹³ is described above. In preferred compounds, R¹³ is NR¹⁴R¹⁵, wherein R¹⁴ and R¹⁵ are independently selected from the group consisting of hydrogen, halo(C₁₋₅)alkyl or C₁₋₅ alkyl. More preferably, wherein R¹⁴ and R¹⁵ are independently selected from the group consisting of hydrogen, methyl and 18Fluoro (C₁₋₅)alkyl.

With respect to the relative positions of any substituent on an aromatic ring, it is envisioned that R¹³ can occur at any available position. The position of R¹³ is independent of the position of any substituent on the opposite ring. In preferred compounds R¹³ is either in a meta or para position relative to the linkage bond between the two aromatic rings. Most preferably, R¹³ is in the para position.

Useful values of R³⁰ are as described above. Preferred values include halogen, C₁₋₅ alkyl, and halo(C₁₋₅)alkyl. Most preferably, R³⁰ is selected from the group consisting of ¹²⁵I, ¹²³I, ¹³¹I, ¹⁸F, ⁷⁶Br, ⁷⁷Br and ¹⁸Fluoro(C₁₋₅)alkyl.

Useful values of R³¹ are as described above. Preferably, R³¹ is C₁₋₅ alkyl. Most preferably, R³¹ is methyl.

The present invention is also directed to compounds of Formula VI: 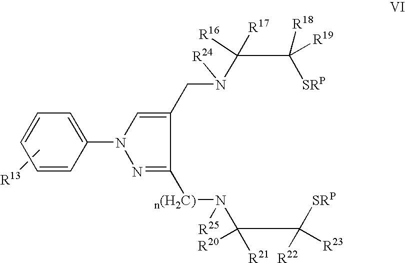
or a pharmaceutically acceptable salt thereof, wherein:

n is zero or one,

R¹³ is selected from the group consisting of:
a. C₁₋₅ alkyl,
b. cyano,
c. trifluoromethyl,
d. nitro,
e. halo(C₁₋₅)alkyl,
f. C₁₋₅ alkylthio,
g. hydroxy(C₁₋₅)alkyl,
h. halogen,
i. halo(C₁₋₅)alkoxy,
j. carboxy(C₁₋₅)alkyl,
k. hydroxy,
l. C₁₋₅ alkoxy,
m. NR¹⁴R¹⁵, wherein
   R¹⁴ and R¹⁵ are independently hydrogen, halo(C₁₋₅)alkyl or C₁₋₅ alkyl,
n. phenyl(C₁₋₅)alkyl,
o. C₆₋₁₀ aryl,
p. heteroaryl,
q. heterocycle,
r. heterocycle(C₁₋₅)alkyl, and
s. C₃₋₆ cycloalkyl,
   wherein said phenyl(C₁₋₅)alkyl, C₆₋₁₀ aryl, heteroaryl, heterocycle, heterocycle(C₁₋₅)alkyl or C₃₋₆ cycloalkyl is substituted with one of the following: C₁₋₅ alkylthio, C₁₋₅ alkylsulfonyl, methoxy, hydroxy, dimethylamino or methylamino,

R¹⁶ R¹⁷, R¹⁸, R¹⁹, R²⁰, R²¹, R²², R²³, R²⁴ and R²⁵ are independently selected from the group consisting of hydrogen, halogen, C₁₋₅ alkyl, cyano, carboxy(C₁₋₅)alkyl, hydroxy(C₁₋₅)alkyl, trifluoromethyl, nitro, methylamino, dimethylamino, halo(C₁₋₅)alkyl, phenyl(C₁₋₅)alkyl, C₃₋₆ cycloalkyl, heterocycle (C₁₋₅)alkyl and carbonyl,

and, R^{P} is a sulfhydryl protecting group such as methoxymethyl, methoxyethoxymethyl, p-methoxybenzyl and benzyl.

Useful compounds falling within the scope of Formula VI are those wherein R¹³, R¹⁶, R¹⁷, R¹⁸, R¹⁹, R²⁰, R²¹, R²², R²³, R²⁴ and R²⁵ above. Particularly useful compounds are those wherein R¹³ is NR¹⁴R¹⁵, wherein R¹⁴ and R¹⁵ are independently hydrogen, halo(C₁₋₅)alkyl or C₁₋₅ alkyl. Preferable values of R¹⁶ and R¹⁷are hydrogen and C₁₋₅ alkyl or R¹⁶ and R¹⁷ are taken together to form a carbonyl. In these preferred compounds, it is more desirable that R¹⁸, R¹⁹, R²⁰, R²¹, R²², R²³, R²⁴ and R²⁵ are independently hydrogen or C₁₋₅ alkyl. In another preferred embodiment, R¹⁶, R¹⁷, R¹⁸, R¹⁹, R²⁰, R²¹, R²², R²³, R²⁴ and R²⁵ are hydrogen.

Useful compounds are those where n equals zero or one. In all embodiments, it is preferred that n equals zero.

With respect to the relative positions of any substituent on an aromatic ring, it is envisioned that R¹³ can occur at any available position. In preferred compounds R¹³ is either in a meta or para position relative to the linkage bond between the two aromatic rings. Most preferably, R¹³ is in the para position.

The tetradentate metal ligand moiety of Formula VI is capable of complexing with a metal, such as 99m-pertechnetate, as described herein to form metal chelate derivatives (radioisotope complex).

The present invention is also directed to radioisotope complex of Formula VI having the following structure: 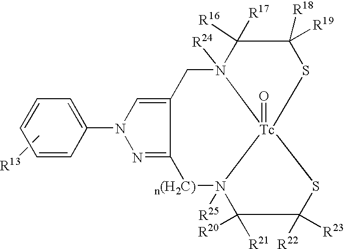
wherein R¹³ is as described for Formula II, and R²⁴ and R²⁵ are as described for the radioisotope complex of Formula III.

It is also to be understood that the present invention is considered to include stereoisomers as well as optical isomers, e.g. mixtures of enantiomers as well as individual enantiomers and diastereomers, which arise as a consequence of structural asymmetry in selected compounds of the present series.

The compounds of Formula I, II, III, IV, V or VI may also be solvated, especially hydrated. Hydration may occur during manufacturing of the compounds or compositions comprising the compounds, or the hydration may occur over time due to the hygroscopic nature of the compounds. In addition, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

When any variable occurs more than one time in any constituent or in Formula I, II, III, IV, V or VI its definition on each occurrence is independent of its definition at every other occurrence. Also combinations of substituents and/or variables are permissible only if such combinations result in stable compounds.

The term “alkyl” as employed herein by itself or as part of another group refers to both straight and branched chain radicals of up to 8 carbons, preferably 6 carbons, more preferably 4 carbons, such as methyl, ethyl, propyl, isopropyl, butyl, t-butyl, and isobutyl.

The term “alkoxy” is used herein to mean a straight or branched chain alkyl radical, as defined above, unless the chain length is limited thereto, bonded to an oxygen atom, including, but not limited to, methoxy, ethoxy, n-propoxy, isopropoxy, and the like. Preferably the alkoxy chain is 1 to 6 carbon atoms in length, more preferably 1-4 carbon atoms in length.

The term “monoalkylamine” as employed herein by itself or as part of another group refers to an amino group which is substituted with one alkyl group as defined above. Thus, the term “methylamino” refers to a neutral group or ring substituent, wherein the N is connected to a compound of one of the general formulas disclosed herein via the ring or a chain of the compound, wherein the N is further bound to a methyl and a hydrogen. Further, the N may be charged and may form salts.

The term “dialkylamine” as employed herein by itself or as part of another group refers to an amino group which is substituted with two alkyl groups as defined above. Thus, the term “dimethylamino” refers to a neutral group or ring substituent, wherein the N is connected to a compound of one of the general formulas disclosed herein via the ring or a chain of the compound, wherein the N is further bound to two methyl groups. Further, the N may be charged and may form salts.

The term “hydroxy(C₁₋₅)alkyl” as employed herein refers to an alkyl chain connected to a compound of one of the general formulas disclosed herein via the ring or a chain of the compound, wherein the distal portion of the alkyl chain of the group contains a hydroxy moiety. The alkyl chain can contain any number of carbons, but preferably the number of carbons in the alkyl chain is from 1 to 5.

The term “halo” employed herein by itself or as part of another group refers to chlorine, bromine, fluorine or iodine.

The term “haloalkyl” as employed herein refers to any of the above alkyl groups substituted by one or more chlorine, bromine, fluorine or iodine with fluorine and chlorine being preferred, such as chloromethyl, iodomethyl, trifluoromethyl, 2,2,2-trifluoroethyl, and 2-chloroethyl.

The term “alkylthio” as employed herein by itself or as part of another group refers to a thioether of the structure: R—S, wherein R is a C₁₋₄ alkyl as defined above.

The term “alkylsulfonyl” as employed herein by itself or as part of another group refers to a sulfone of the structure: R—SO₂, wherein R is a C₁₋₄ alkyl as defined above.

The term “aryl” as employed herein by itself or as part of another group refers to monocyclic or bicyclic aromatic groups containing from 6 to 12 carbons in the ring portion, preferably 6-10 carbons in the ring portion, such as phenyl, naphthyl or tetrahydronaphthyl.

The term “heterocycle” or “heterocyclic ring”, as used herein except where noted, represents a stable 5- to 7-membered mono-heterocyclic ring system which may be saturated or unsaturated, and which consists of carbon atoms and from one to three heteroatoms selected from the group consisting, of N, O, and S, and wherein the nitrogen and sulfur heteroatom may optionally be oxidized. Especially useful are rings contain one nitrogen combined with one oxygen or sulfur, or two nitrogen heteroatoms. Examples of such heterocyclic groups include piperidinyl, pyrrolyl, pyrrolidinyl, imidazolyl, imidazinyl, imidazolidinyl, pyridyl, pyrazinyl, pyrimidinyl, oxazolyl, oxazolidinyl, isoxazolyl, isoxazolidinyl, thiazolyl, thiazolidinyl, isothiazolyl, homopiperidinyl, homopiperazinyl, pyridazinyl, pyrazolyl, and pyrazolidinyl, most preferably thiamorpholinyl, piperazinyl, and morpholinyl.

The term “heteroatom” is used herein to mean an oxygen atom (“O”), a sulfur atom (“S”) or a nitrogen atom (“N”). It will be recognized that when the heteroatom is nitrogen, it may form an NR^{a}R^{b} moiety, wherein R^{a} and R^{b} are, independently from one another, hydrogen or C₁₋₄ alkyl, C₂₋₄ aminoalkyl, C₁₋₄ halo alkyl, halo benzyl, or R¹ and R² are taken together to form a 5- to 7-member heterocyclic ring optionally having O, S or NR^{c} in said ring, where R^{c} is hydrogen or C₁₋₄ alkyl.

The term “heteroaryl” as employed herein refers to groups having 5 to 14 ring atoms; 6, 10 or 14 n electrons shared in a cyclic array; and containing carbon atoms and 1, 2 or 3 oxygen, nitrogen or sulfur heteroatoms (where examples of heteroaryl groups are: thienyl, benzo[b]thienyl, naphtho[2,3-b]thienyl, thianthrenyl, furyl, pyranyl, isobenzofuranyl, benzoxazolyl, chromenyl, xanthenyl, phenoxathiinyl, 2H-pyrrolyl, pyrrolyl, imidazolyl, pyrazolyl, pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalazinyl, naphthyridinyl, quinazolinyl, cinnolinyl, pteridinyl, 4aH-carbazolyl, carbazolyl, β-carbolinyl, phenanthridinyl, acridinyl, perimidinyl, phenanthrolinyl, phenazinyl, isothiazolyl, phenothiazinyl, isoxazolyl, furazanyl and phenoxazinyl groups).

The term “aralkyl” or “arylalkyl” as employed herein by itself or as part of another group refers to C₁₋₆alkyl groups as discussed above having an aryl substituent, such as benzyl, phenylethyl or 2-naphthylmethyl.

Another aspect of this invention is related to methods of preparing compounds of Formula I, II, III, IV, V or VI.

In embodiments of Formula III, the groups R^{P} are both hydrogen, or can be any of the variety of protecting groups available for sulfur, including methoxymethyl, methoxyethoxymethyl, p-methoxybenzyl or benzyl. Sulfur protecting groups are described in detail in Greene, T. W. and Wuts, P. G. M., Protective Groups in Organic Synthesis, 2nd Edition, John Wiley and Sons, Inc., New York (1991). Protecting group R^{P} can be removed by appropriate methods well known in the art of organic synthesis, such as trifluoroacetic acid, mercuric chloride or sodium in liquid ammonia. In the case of Lewis acid labile groups, including acetamidomethyl and benzamidomethyl, R^{P} can be left intact. Labeling of the ligand with technetium in this case will cleave the protecting group, rendering the protected diaminedithiol equivalent to the unprotected form.

Tc-99m complexes can be prepared as follows. A small amount of non-radiolabeled compound (1-2 mg) is dissolved in 100 μL EtOH and mixed with 200 μL HCl (1 N) and 1 mL Sn-glucoheptonate solution (containing 8-32 μg SnCl₂ and 80-320 μg Na-glucoheptonate, pH 6.67) and 50 μL EDTA solution (0.1 N). [^{99m}Tc]Pertechnetate (100-200 μL; ranging from 2-20 mCi) saline solution are then added. The reaction is heated for 30 min at 100° C., then cooled to room temperature. The reaction mixture is analyzed on TLC (EtOH:conc. NH3 9:1) for product formation and purity check. The mixture can be neutralized with phosphate buffer to pH 5.0.

The present invention further relates to a method of preparing a technetium-99m complex according to the present invention by reacting technetium-99m in the form of a pertechnetate in the presence of a reducing agent and optionally a suitable chelator with an appropriate Ch-containing compound.

The reducing agent serves to reduce the Tc-99m perteclnetate which is eluted from a molybdenum-technetium generator in a physiological saline solution. Suitable reducing agents are, for example, dithionite, formamidine sulphinic acid, diaminoethane disulphinate or suitable metallic reducing agents such as Sn(II), Fe(II), Cu(I), Ti(III) or Sb(III). Sn(II) has proven to be particularly suitable.

For the above-mentioned complex-forming reaction, technetium-99m is reacted with an appropriate compound of the invention as a salt or in the form of technetium bound to comparatively weak chelators. In the latter case the desired technetium-99m complex is formed by ligand exchange. Examples of suitable chelators for the radionuclide are dicarboxylic acids, such as oxalic acid, malonic acid, succinic acid, maleic acid, orthophtalic acid, malic acid, lactic acid, tartaric acid, citric acid, ascorbic acid, salicylic acid or derivatives of these acids; phosphorus compounds such as pyrophosphates; or enolates. Citric acid, tartaric acid, ascorbic acid, glucoheptonic acid or a derivative thereof are particularly suitable chelators for this purpose, because a chelate of technetium-99m with one of these chelators undergoes the desired ligand exchange particularly easily.

The most commonly used procedure for preparing [Tc^{v}O]⁺³N₂S₂ complexes is based on stannous (II) chloride reduction of [^{99m}Tc]pertechnetate, the common starting material. The labeling procedure normally relies on a Tc-99m ligand exchange reaction between Tc-99m (Sn)-glucoheptonate and the N₂S₂ ligand. Preparation of stannous (II) chloride and preserving it in a consistent stannous (II) form is critically important for the success of the labeling reaction. To stabilize the air-sensitive stannous ion it is a common practice in nuclear medicine to use a lyophilized kit, in which the stannous ion is in a lyophilized powder form mixed with an excess amount of glucoheptonate under an inert gas like nitrogen or argon. The preparation of the lyophilized stannous chloride/sodium glucoheptonate kits ensures that the labeling reaction is reproducible and predictable. The N₂S₂ ligands are usually air-sensitive (thiols are easily oxidized by air) and there are subsequent reactions which lead to decomposition of the ligands. The most convenient and predictable method to preserve the ligands is to produce lyophilized kits containing 100-500 μg of the ligands under argon or nitrogen.

The present invention is further directed to methods of preparing compounds of the above Formula I, II, III, IV and V. All reagents used in the synthesis were commercial products and were used without further purification unless otherwise indicated. Anhydrous Na₂SO₄ was used as a drying agent. Flash column chromatography was performed on 230-400 mesh silica gel.

The compounds of this invention can be prepared by reactions described in Schemes 1-6. Synthesis of N,N-dimethylamino derivatives of fluorene was successfully achieved by a reductive methylation reaction shown in Scheme 1. Starting with 2- or 3-aminofluorenes, 1a-1f, the amino group was converted to the N,N-dimethylamino group (2a-2f) in excellent yield (>90%) using paraformaldehyde in the presence of sodium cyanoborohydride as a reducing agent (4). A same reaction was applied in the methylation of 9-fluorenones (Scheme 2) by which the amino-9-fluorenones were converted to N,N-dimethylamino-9-hydroxyfluorenes (3a-3d) in good yield (>80%). Under the reductive methylation condition, the keto group of fluorenone was reduced to 9-hydroxy group. To preserve the keto group of the 9-fluorenone, an alternative method was employed for the methylation reaction. Using methyliodide/K₂CO₃ in refluxing acetonitrile, the amino group of 9-fluorenone was methylated to the N,N-dimethylamino-9-fluorenones (4a-4d) (Scheme 3). The yields for this methylation reaction were less predictable (ranging from 18-70%). To prepare the tri-butyltin, 5, the bromo derivative, 2d, was treated with bis(tributyltin) and Pd(Ph₃P)₂ in a mixed solvent of dioxane:triethylamine at 90° C. to give the desired tributyltin derivative, 5. Preparation of radioiodinated [¹²⁵I]2f was carried out by an iododestannylation reaction of 5, which was catalyzed by hydrogen peroxide (Scheme 3). The desired tracer, [¹²⁵I]2f, was readily purified by HPLC (radiochemical yield 80%, radiochemical purity >95%). Scheme 4 depicts a synthetic route for preparing fluorene compounds of Formula II. Scheme 5 and 6 depict a synthetic route for preparing biphenyl compounds of Formula I. Schemes 7 and 8 depict a synthetic route for preparing metal chelated biphenyl compounds of Formula III, including the stable Tc-99m labeled compound ^{[99mTC]}35. Scheme 9 depicts a synthetic route for pyrazole derivatives of Formula V. Arylation of a starting material, 4-bromo-3-methylpyrazole, was carried out with 4-fluoronitrobenzene using t-BuOK as base in DMSO at 75° C. for 1 h, and a 9:1 mixture of regioisomers, compound 23 and 3-bromo-1-(4-nitrophenyl)-4-methylpyrazole, was obtained. The two isomers were successfully separated by a careful chromatographic purification. After purification, the yield of compound 23 and the isomer was 65 and 5%, respectively. Conversion of 23 to the dimethylamino derivative, 25, was achieved by reduction of the nitro group to amino group with SnCl₂ (yield 85%) and subsequent dimethylation of the amino group (yield 68%). The required selective dimethylation of the amino group was accomplished by a method previously reported (Gribble, 1987). When compound 25 was reacted with Bu₃SnCl using BuLi as base the corresponding tributyltin derivative, 26, was obtained in yields of 35%. The tributyltin derivative, 26, was reacted with iodine in chloroform at room temperature to give the iodo derivative, 27, in yields of 55%. Conversion of 24 to the monomethylarnino derivative 28 was achieved by a method previously reported (Barluenga, 1984). Compound 31 and 34 were also obtained by the reduction and selective dimethylation reaction described above.

Scheme 1

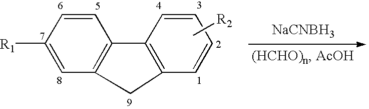

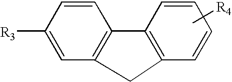

| | R₁ | R₂ | Ki, nM |
|---|---|---|---|
| 1a | H | 2-NH₂ | >1,000 |
| 1b | H | 3-NH₂ | >1,000 |
| 1c | H | 4-NH₂ | >1,000 |
| 1d | Br | 2-NH₂ | 56 ± 2 |
| 1e | NH₂ | 2-NH₂ | >1,000 |
| 1f | I | 2-NH₂ | — |
| | R₃ | R₄ | Ki, nM |
| 2a | H | 2-NMe₂ | >1000 |
| 2b | H | 3-NMe₂ | 23.5 ± 6.0 |
| 2c | H | 4-NMe₂ | >1,000 |
| 2d | Br | 2-NMe₂ | 0.85 ± 0.1 |
| 2e | NMe₂ | 2-NMe₂ | 15.4 ± 5.0 |
| 2f | I | 2-NMe₂ | 0.92 ± 0.10 |

Scheme 2

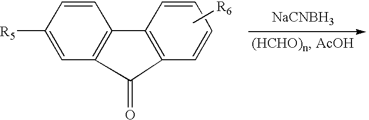

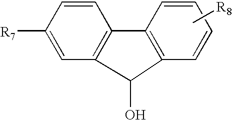

| R₅ | R₆ |
|---|---|
| H | 2-NH₂ |
| H | 4-NH₂ |
| Br | 2-NH₂ |
| H | 2-NH₂ and 3-Br |

| | R₇ | R₈ | Ki, nM |
|---|---|---|---|
| 3a | H | 2-NMe₂ | >1,000 |
| 3b | H | 4-NMe₂ | >1,000 |
| 3c | Br | 2-NMe₂ | 88 ± 4 |
| 3d | H | 2-NMe₂ and 3-Br | >1,000 |

Scheme 3

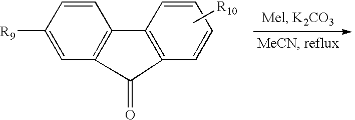

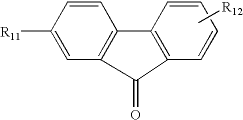

| R₉ | R₁₀ |
|---|---|
| H | 2-NH₂ |
| H | 3-NH₂ |
| H | 4-NH₂ |
| Br | 2-NH₂ |

| | R₁₁ | R₁₂ | Ki, nM |
|---|---|---|---|
| 4a | H | 2-NMe₂ | >1,000 |
| 4b | H | 3-NMe₂ | >1,000 |
| 4c | H | 4-NMe₂ | >1,000 |
| 4d | Br | 2-NMe₂ | 16.5 ± 4 |

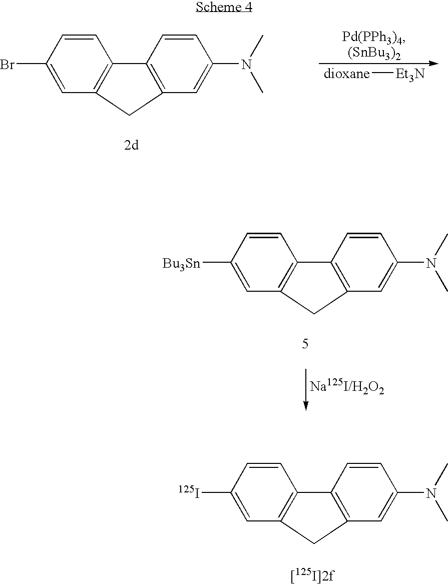

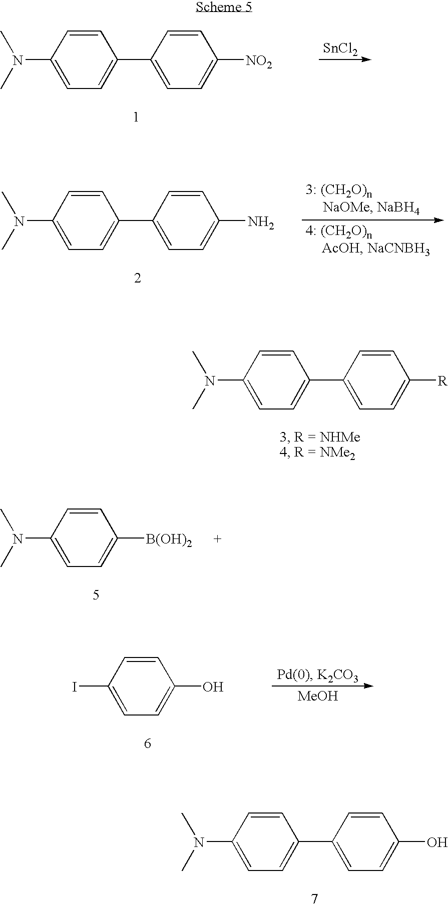

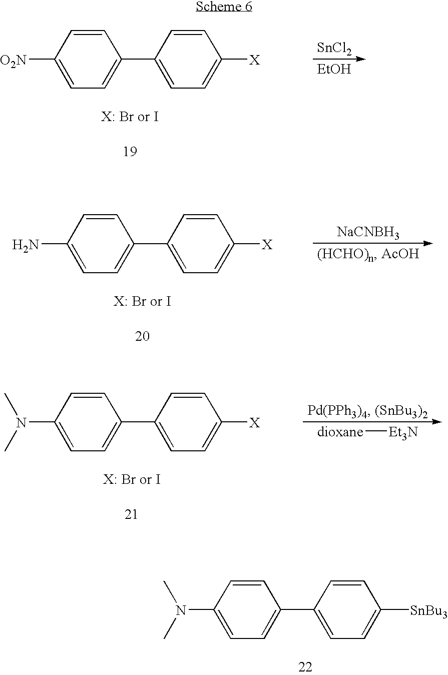

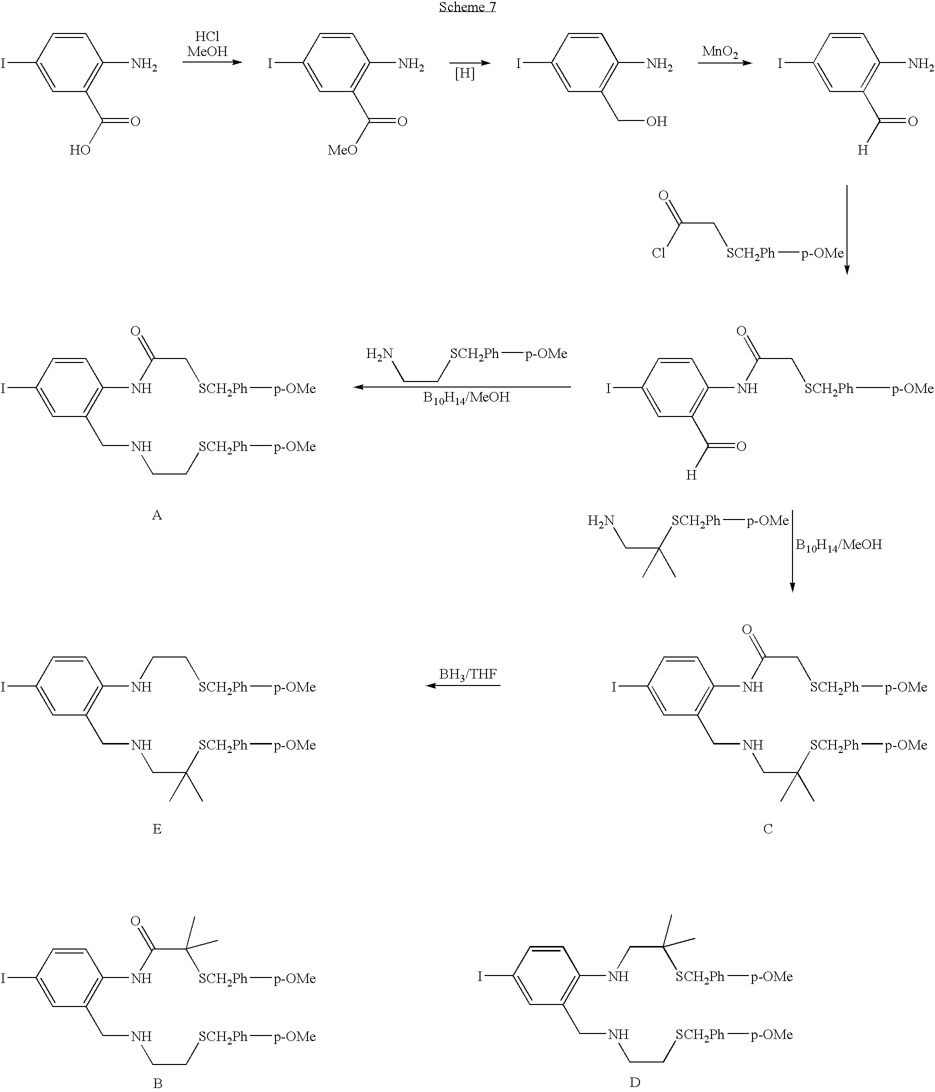

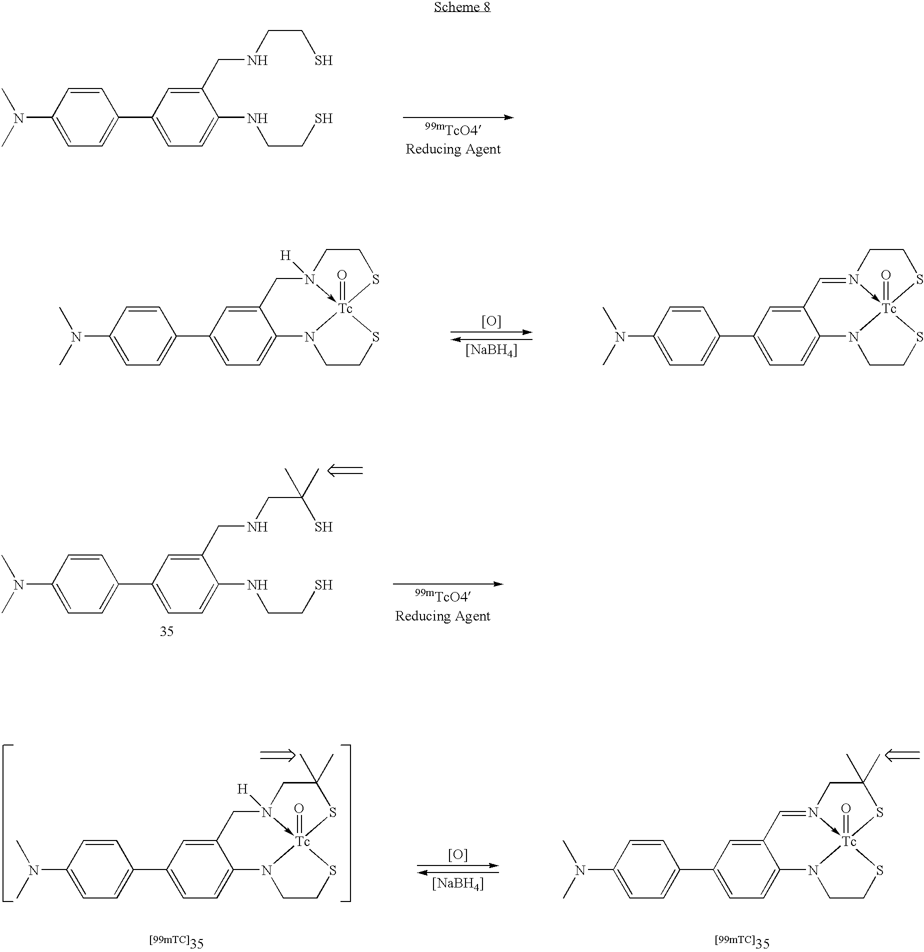

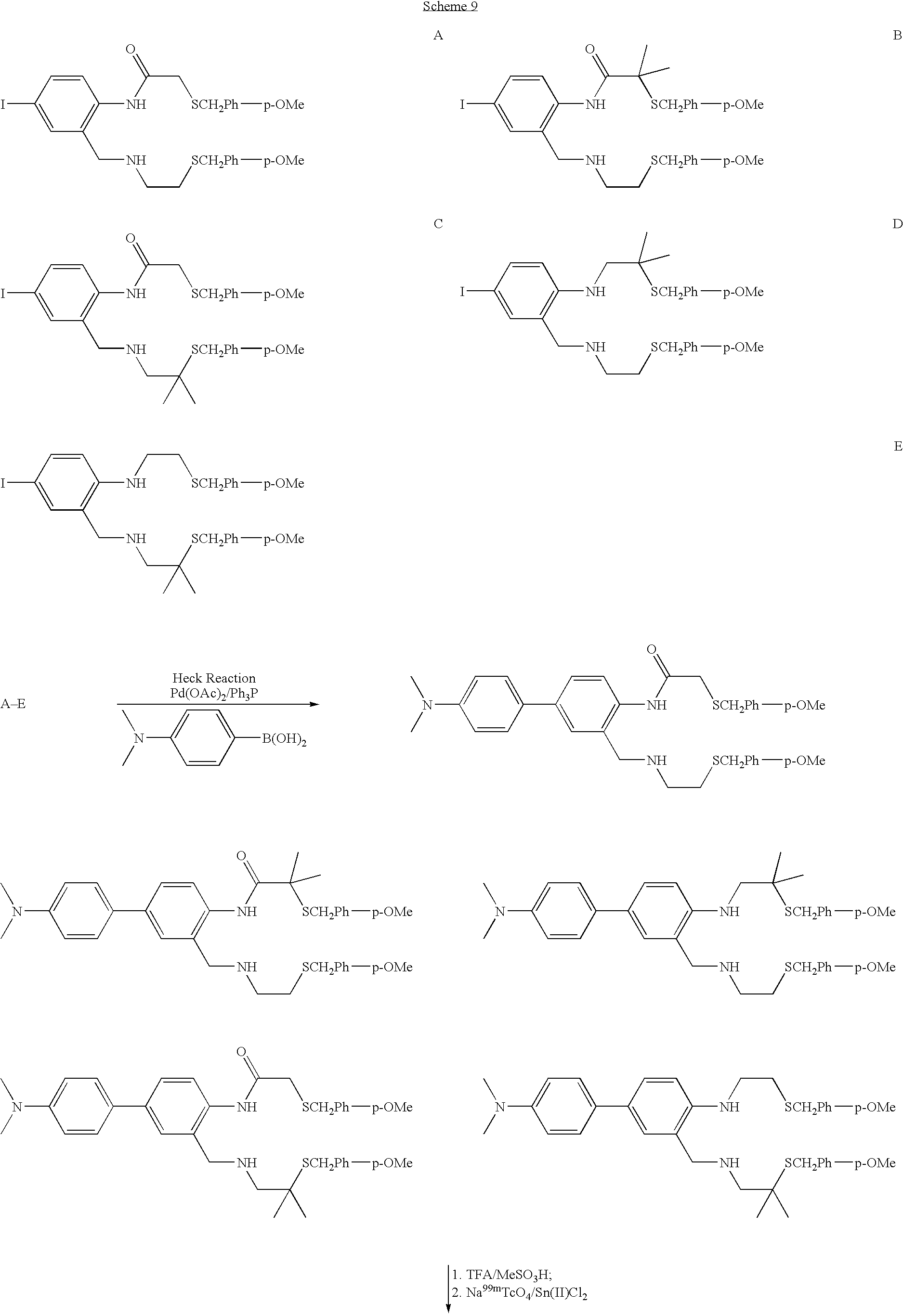

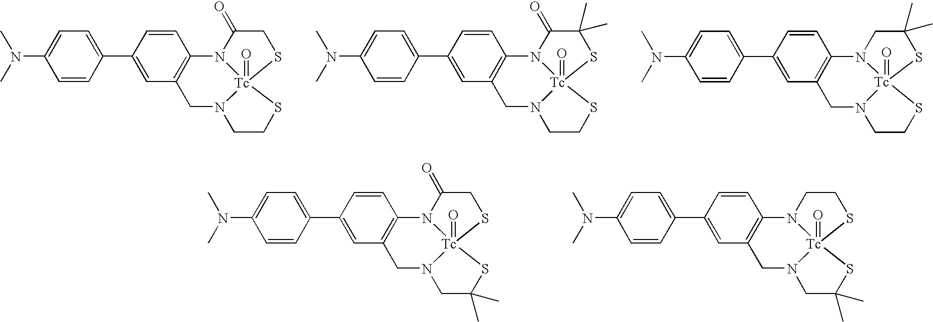

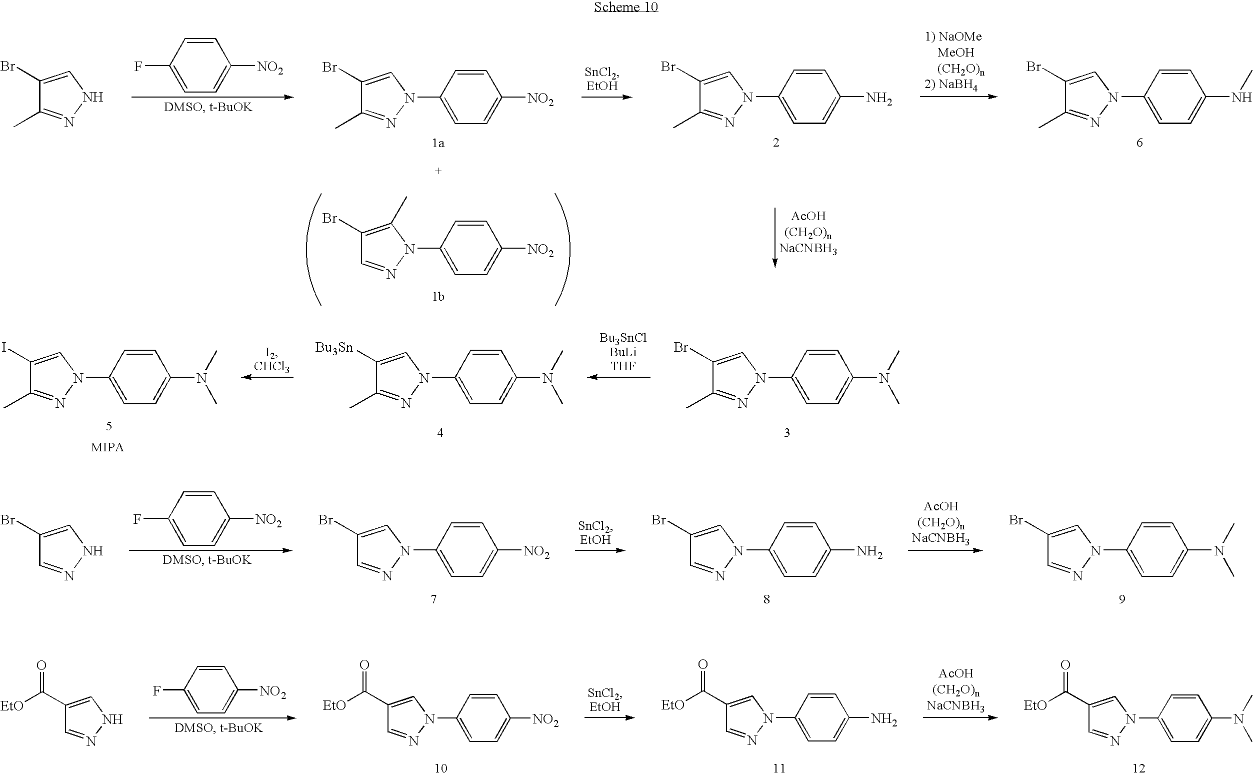

When the compounds of this invention are to be used as imaging agents, they must be labeled with suitable radioactive halogen isotopes. Although ¹²⁵I-isotopes are useful for laboratory testing, they will generally not be useful for actual diagnostic purposes because of the relatively long half-life (60 days) and low gamma-emission (30-65 Kev) of ¹²⁵I. The isotope ¹²³I has a half life of thirteen hours and gamma energy of 159 KeV, and it is therefore expected that labeling of ligands to be used for diagnostic purposes would be with this isotope. Other isotopes which may be used include ¹³¹I (half life of 2 hours). Suitable bromine isotopes include ⁷⁷Br and ⁷⁶Br.

The radiohalogenated compounds of this invention lend themselves easily to formation from materials which could be provided to users in kits. Kits for forming the imaging agents can contain, for example, a vial containing a physiologically suitable solution of an intermediate of Formula I, II, III, IV, V or VI in a concentration and at a pH suitable for optimal complexing conditions. The user would add to the vial an appropriate quantity of the radioisotope, e.g., Na¹²³I, and an oxidant, such as hydrogen peroxide. The resulting labeled ligand may then be administered intravenously to a patient, and receptors in the brain imaged by means of measuring the gamma ray or photo emissions therefrom.

Since the radiopharmaceutical composition according to the present invention can be prepared easily and simply, the preparation can be carried out readily by the user. Therefore, the present invention also relates to a kit, comprising:

(1) A non-radiolabeled compound of the invention, the compound optionally being in a dry condition; and also optionally having an inert, pharmaceutically acceptable carrier and/or auxiliary substances added thereto; and

(2) a reducing agent and optionally a chelator;

wherein ingredients (1) and (2) may optionally be combined; and further wherein instructions for use with a prescription for carrying out the above-described method by reacting ingredients (1) and (2) with technetium-99m in the form of a pertechnetate solution may be optionally included.

Examples of suitable reducing agents and chelators for the above kit have been listed above. The pertechnetate solution can be obtained by the user from a molybdenum-technetium-generator. Such generators are available in a number of institutions that perform radiodiagnostic procedures. As noted above the ingredients (1) and (2) may be combined, provided they are compatible. Such a monocomponent kit, in which the combined ingredients are preferably lyophilized, is excellently suitable to be reacted by the user with the pertechnetate solution in a simple manner.

When desired, the radioactive diagnostic agent may contain any additive such as pH controlling agents (e.g., acids, bases, buffers), stabilizers (e.g., ascorbic acid) or isotonizing agents (e.g., sodium chloride).

The term “pharmaceutically acceptable salt” as used herein refers to those carboxylate salts or acid addition salts of the compounds of the present invention which are, within the scope of sound medical judgement, suitable for use in contact with the tissues of patients without undue toxicity, irritation, allergic response, and the like, commensurate with a reasonable benefit/risk ratio, and effective for their intended use, as well as the zwitterionic forms, where possible, of the compounds of the invention. The term “salts” refers to the relatively nontoxic, inorganic and organic acid addition salts of compounds of the present invention. Also included are those salts derived from non-toxic organic acids such as aliphatic mono and dicarboxylic acids, for example acetic acid, phenyl-substituted alkanoic acids, hydroxy alkanoic and alkanedioic acids, aromatic acids, and aliphatic and aromatic sulfonic acids. These salts can be prepared in situ during the final isolation and purification of the compounds or by separately reacting the purified compound in its free base form with a suitable organic or inorganic acid and isolating the salt thus formed. Further representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactiobionate and laurylsulphonate salts, propionate, pivalate, cyclamate, isethionate, and the like. These may include cations based on the alkali and alkaline earth metals, such as sodium, lithium, potassium, calcium, magnesium, and the like, as well as, nontoxic ammonium, quaternary ammonium and amine cations including, but not limited to ammonium, tetramethylammonium, tetraethylammonium, methylamine, dimethylamine, trimethylamine, triethylamine, ethylamine, and the like. (See, for example, Berge S. M., et al;, Pharmaceutical Salts, J Pharm. Sci. 66:1-19 (1977) which is incorporated herein by reference.)

In the first step of the present method of imaging, a labeled compound of Formula I, II, III, IV, V or VI is introduced into a tissue or a patient in a detectable quantity. The compound is typically part of a pharmaceutical composition and is administered to the tissue or the patient by methods well known to those skilled in the art.

For example, the compound can be administered either orally, rectally, parenterally (intravenous, by intramuscularly or subcutaneously), intracisternally, intravaginally, intraperitoneally, intravesically, locally (powders, ointments or drops), or as a buccal or nasal spray.

In a preferred embodiment of the invention, the labeled compound is introduced into a patient in a detectable quantity and after sufficient time has passed for the compound to become associated with amyloid deposits, the labeled compound is detected noninvasively inside the patient. In another embodiment of the invention, a labeled compound of Formula I, II, III, IV, V or VI is introduced into a patient, sufficient time is allowed for the compound to become associated with amyloid deposits, and then a sample of tissue from the patient is removed and the labeled compound in the tissue is detected apart from the patient. In a third embodiment of the invention, a tissue sample is removed from a patient and a labeled compound of Formula I, II, III, IV, V or VI is introduced into the tissue sample. After a sufficient amount of time for the compound to become bound to amyloid deposits, the compound is detected.

The administration of the labeled compound to a patient can be by a general or local administration route. For example, the labeled compound may be administered to the patient such that it is delivered throughout the body. Alternatively, the labeled compound can be administered to a specific organ or tissue of interest. For example, it is desirable to locate and quantitate amyloid deposits in the brain in order to diagnose or track the progress of Alzheimer's disease in a patient.

The term “tissue” means a part of a patient's body. Examples of tissues include the brain, heart, liver, blood vessels, and arteries. A detectable quantity is a quantity of labeled compound necessary to be detected by the detection method chosen. The amount of a labeled compound to be introduced into a patient in order to provide for detection can readily be determined by those skilled in the art. For example, increasing amounts of the labeled compound can be given to a patient until the compound is detected by the detection method of choice. A label is introduced into the compounds to provide for detection of the compounds.

The term “patient” means humans and other animals. Those skilled in the art are also familiar with determining the amount of time sufficient for a compound to become associated with amyloid deposits. The amount of time necessary can easily be determined by introducing a detectable amount of a labeled compound of Formula I, II, III, IV, V or VI into a patient and then detecting the labeled compound at various times after administration.

The term “associated” means a chemical interaction between the labeled compound and the amyloid deposit. Examples of associations include covalent bonds, ionic bonds, hydrophilic-hydrophilic interactions, hydrophobic-hydrophobic interactions, and complexes.

Those skilled in the art are familiar with the various ways to detect labeled compounds. For example, magnetic resonance imaging (MRI), positron emission tomography (PET), or single photon emission computed tomography (SPECT) can be used to detect radiolabeled compounds. The label that is introduced into the compound will depend on the detection method desired. For example, if PET is selected as a detection method, the compound must possess a positron-emitting atom, such as ¹¹C or ¹⁸F.

The radioactive diagnostic agent should have sufficient radioactivity and radioactivity concentration which can assure reliable diagnosis. For instance, in case of the radioactive metal being technetium-99m, it may be included usually in an amount of 0.1 to 50 mCi in about 0.5 to 5.0 ml at the time of administration. The amount of a compound of Formula I, II, III, IV, V or VI may be such as sufficient to form a stable chelate compound with the radioactive metal.

The thus formed chelate compound as a radioactive diagnostic agent is sufficiently stable, and therefore it may be immediately administered as such or stored until its use. When desired, the radioactive diagnostic agent may contain any additive such as pH controlling agents (e.g., acids, bases, buffers), stabilizers (e.g., ascorbic acid) or isotonizing agents (e.g., sodium chloride).

The imaging of amyloid deposits can also be carried out quantitatively so that the amount of amyloid deposits can be determined.

Preferred compounds for imaging include a radioisotope such as ¹²³I, ¹²⁵I, ¹³¹, ¹⁸F, ⁷⁶Br, or ⁷⁷Br.

The present invention is also directed at a method of imaging amyloid deposits. One of the key prerequisites for an iii vivo imaging agent of the brain is the ability to cross the intact blood-brain barrier after a bolus iv injection.

Another aspect of the invention is a method of inhibiting amyloid plaque aggregation. The present invention also provides a method of inhibiting the aggregation of amyloid proteins to form amyloid deposits, by administering to a patient an amyloid inhibiting amount of a compound of the above Formula I, II, III, IV, V or VI.

Those skilled in the art are readily able to determine an amyloid inhibiting amount by simply administering a compound of Formula I, II, III, IV, V or VI to a patient in increasing amounts until the growth of amyloid deposits is decreased or stopped. The rate of growth can be assessed using imaging as described above or by taking a tissue sample from a patient and observing the amyloid deposits therein. The compounds of the present invention can be administered to a patient at dosage levels in the range of about 0. 1 to about 1,000 mg per day. For a normal human adult having a body weight of about 70 kg, a dosage in the range of about 0.01 to about 100 mg per kilogram of body weight per day is sufficient. The specific dosage used, however, can vary. For example, the dosage can depend on a number of factors including the requirements of the patient, the severity of the condition being treated, and the pharmacological activity of the compound being used. The determination of optimum dosages for a particular patient is well known to those skilled in the art.

The following examples are illustrative, but not limiting, of the method and compositions of the present invention. Other suitable modifications and adaptations of the variety of conditions and parameters normally encountered and obvious to those skilled in the art are within the spirit and scope of the invention.

### EXAMPLE 1

### 2-(Dimethylamino)fluorene (2a)

To a stirred mixture of 2-aminofluorene (119 mg, 0.66 mmol) and papraformaldehyde (300 mg, 10 mmol) in 5 ml of AcOH at room temperature was added in one portion of NaCNBH₃ (300. mg, 4.8 mmol). The resulting mixture was stirred at room temperature for 18 h, then carefully poured into 25% aq. NaOH and ice chips to make strongly alkaline (pH 11) and extracted with methylene chloride. The combined extracts were dried, filtered, and concentrated in vacuo. The residue was subjected to flash chromatography (EtOAc: Hex, 1:4). and gave 129 mg of 2-(dimethylamino)fluorene (94%). ¹H NMR (200 MHz, CDCl₃): δ 3.09 (s, 6H), 7.00 (d, J=7.55 Hz, 1H), 7.33-7.38 (m, 2H), 7.61-7.96 (m, 5H), 8.11 (d, J=8.40 Hz, 1H); ¹³C NMR (50 MHz, CDCl₃): 6 45.43, 114.04, 119.74, 120.60, 120.98, 121.30, 123.99, 125.34, 126.20, 126.45, 127.40, 130.49, 134.03, 137.26, 139.08, 139.39, 151.83.

### EXAMPLE 2

### 3-(Dimethylamino)fluorene (2b)

The same reaction as described above for preparing 2a was employed, and 2b was obtained in 93% from 3-aminofluorene. ¹H NMR (200 MHz, CDCl₃): δ 3.05 (s, 6H), 3.89 (s, 2H), 6.82 (dd, J=8.44 Hz, J=2.35 Hz, 1H), 6.99 (s, 1H), 7.18-7.25 (m, 1H), 7.36 (t, J=7.30 Hz, 1H), 7.51 (d, J=7.31 Hz, 1H), 7.68 (d, J=8.44 Hz, 2H); ¹³C NMR (50 MHz, CDCl₃): δ 37.14, 41.04, 109.28, 111.67, 118.52, 120.43, 124.71, 124.79, 126.64, 131.12, 142.33, 142.43, 145.02, 150.33; Anal. Calcd for C₁₅H₁₅N: C, 86.08; H, 7.22. Found: C, 86.46; H, 6.89.

### EXAMPLE 3

### 4-(Dimethylamino)fluorene (2c)

The same reaction as described above for preparing 2a was employed, and 2c was obtained in 94% from 4-aminofluorene. ¹H NMR (200 MHz, CDCl₃): δ 3.01 (s, 6H), 4.01 (s, 2H), 6.96 (d, J=7.78 Hz, 1H), 7.33-7.54 (m, 4H), 7.63 (d, J=6.99 Hz, 5H), 7.85 (d, J=6.96 Hz, 1H); ¹³C NMR (50 MHz, CDCl₃): δ 36.99, 43.18, 113.26, 115.20, 119.90, 124.72, 126.62, 128.05, 133.73, 141.90, 143.20, 150.40.

### EXAMPLE 4

### 2-Dimethylamino-7-bromofluorene (2d)

The same reaction as described above for preparing 2a was employed, and 2d was obtained in 95% from 2-amino-7-bromofluorene. ¹H NMR (200 MHz, CDCl₃): δ 3.02 (s, 6H), 3.80 (s, 2H), 6.76 (dd, J=8.47 Hz, J=2.33 Hz, 1H), 6.90 (s, 1H), 7.56-7.61 (m, 4H); 13C NMR (50 MHz, CDCl₃): δ 36.91, 40.90, 108.94, 111.66, 118.14, 119.64, 120.53, 127.84, 129.63, 129.85, 141.44, 144.34, 144.73, 150.50; HRMS: m/z Calcd for C₁₅H₁₄BrN: 287.0301; Found: 287.0282; Anal. Calcd for C₁₅H₁₄BrN: C, 62.52; H, 4.90. Found: C, 62.46; H, 4.90.

### EXAMPLE 5

### 2,7-Bis(dimethylamine)fluorene (2e)

The same reaction as described above for preparing 2a was employed, and 2e was obtained in 61% from 2,7-diaminofluorene. ¹H NMR (200 MHz, CDCl₃): δ 2.98 (s, 6H), 3.81 (s, 2H), 6.75 (dd, J=2.41 Hz, J =8.39 Hz, 2H), 6.94 (s, 2H), 7.50 (d, J=8.37 Hz, 2H); ¹³C NNM (50 MHz, CDCl₃): δ 37.30, 41.30, 109.90, 111.82, 119.00, 132.12, 143.97, 149.23; ERMS: m/z Calcd for C₁₇H₂₀N₂: 252.1626; Found: 252.1618.

### EXAMPLE 6

### 2-Dimethylamino-7-iodofluorene (2f)

The same reaction as described above for preparing 2a was employed, and 2f was obtained in 93% from 2-amino-7-iodofluorene, which is readily prepared by reduction of 2-nitro-7-iodofluorene by SnCl₂. ¹H NMR (200 MHz, CDCl₃): δ 3.01 (s, 6H), 3.81 (s, 2H), 6.76 (dd, J=8.47 Hz, J=2.33 Hz, 1H), 6.90 (s, 1H), 7.56-7.61 (m, 4H); ¹³C NMR (50 MHz, CDCl₃): δ 36.91, 40.90, 108.94, 111.66, 118.14, 119.64, 120.53, 127.84, 129.63, 129.85, 141.44, 144.34, 144.73, 150.50; HRMS: m/z Calcd for C₁₅H₁₄BrN: 335.0171; Found: 335.0184.

### EXAMPLE 7

### 2-Dimethylamino-9-hydroxyfluorene (3a)

To a stirred mixture of 2-amino-9-fluorenone (104 mg, 0.53 mmol) and papraformaldehyde (200 mg, 6 mmol) in 5 ml of AcOH at room temperature was added in one portion of NaCNBH₃ (200 mg, 3.2 mmol). The resulting mixture was stirred at room temperature for 18 h, then carefully poured into 25% aq. NaOH and ice chips to make strongly alkaline (pH 11) and extracted with methylene chloride. The combine extracts were dried, filtered, and concentrated in vacuo. The residue was subjected to flash chromatography (EtOAc: Hex, 1:4) and gave 100 mg of 2-dimethylamino-9-fluorenone (84%). ¹H NMR (200 MHz, CDCl₃): δ 2.08 (br s, 1H), 2.98 (s, 6H), 5.47 (br s, 1H), 6.68 (dd, J=8.41 Hz, J=2.45 Hz, 1H), 7.01 (d, J=2.35 Hz, 1H), 7.18 (dt, J=1.17 Hz, J=7.35 Hz, 1H), 7.32 (dt, J=1.02 Hz, J=7.33 Hz, 1H), 7.44-7.57 (m, 3H); ¹³C NMR (50 MHz, CDCl₃): δ 40.83, 75.35, 109.32, 112.91, 118.52, 120.60, 124.78, 125.69, 128.64, 128.90, 140.78, 144.92, 147.35, 150.83.

### EXAMPLE8

### 4-Dimethylamino-9-hydroxyfluorene (3b)

The same reaction as described above for preparing 3a was employed, and 3b was obtained in 99% from 4-amino-9-fluorenone. ¹H NMR (200 MHz, CDCl₃): δ 2.35 (d, J=9.59 Hz, 1H), 2.83 (s, 6H), 5.46 (d, J=9.44 Hz, 1H), 7.06-7.10 (m, 1H), 7.25-7.44 (m, 4H), 7.58 (d, J=7.26 Hz, 1H), 7.99 (d, J=7.61 Hz, 1H); ¹³C NMR (50 MHz, CDCl₃): δ 44.24, 75.01, 118.41, 118.98, 123.74, 124.33, 126.70, 128.38, 128.78, 132.13, 139.41, 145.61, 147.86, 149.95.

### EXAMPLE 9

### 2-Dimethylamino-7-bromo-9-hydroxyfluorene (3c)

The same reaction as described above for preparing 3a was employed, and 3c was obtained in 87% from 2-amino-7-bromo-9-fluorenone. ¹H NMR (200 MHz, CDCl₃): δ 1.89 (d, J=10.07 Hz, 1H), 3.01 (s, 1H), 5.46 (d, J=9.06 Hz, 1H), 6.71 (dd, J=8.45 Hz, J=2.46 Hz, 1H), 7.00 (d, J=2.41 Hz, 1H), 7.31-7.47 (m, 3H), 7.67 (t, J=1.01 Hz, 1H); ¹³C NMR (50 MHz, CDCl₃): δ 40.63, 74.98, 108.89, 112.82, 118.95, 119.70, 120.68, 127.27, 128.07, 131.78, 139.69, 146.76, 146.98, 150.97; HRMS: m/z Calcd for C₁₅H₁₄BrNO: 303.0259; Found: 303.0242; Anal. Calcd for C₁₅H₁₄BrNO: C, 59.23; H, 4.64. Found: C, 59.41; H, 4.60.

### EXAMPLE 10

### 2-Dimethylamino-3-bromo-9-hydroxyfluorene (3d)

The same reaction as described above for preparing 3a was employed, and 3d was obtained in 85% from 2-amino-3-brorno-9-fluorenone. ¹H NMR (200 MHz, CDCl₃): δ 2.04 (d, J=9.63 Hz, 1H), 2.83 (s, 6H), 5.44 (d, J=9.78 Hz, 1H), 7.25-7.39 (m, 3H), 7.48-7.59 (m, 2H), 7.78 (s, 1H); ¹³C NMR (50 MHz, CDCl₃): δ 44.39, 74.88, 117.21, 119.57, 119.93, 125.03, 125.35, 127.42, 129.18, 135.85, 138.91, 145.53, 145.84, 151.60.

### EXAMPLE 11

### 2-Dimethylamino-9-fluorenone (4a)

To a stirred mixture of 2-amino-9-fluorenone (315 mg, 1.6 mmol) and potassium carbonate (300 mg) in 5 ml of acetonitrile was added in one portion of iodomethane (0.5 ml). After overnight at reflux, NH₄Cl solution (saturated, 5 mL) is added and the mixture is extracted with CH₂Cl₂ (3 Y 30 mL). The combined organic extract is dried over Na₂SO₄, evaporated and purified by flash chromatography (EtOAc: Hex, 1:9) to give 2-dimethylamino-9-fluorenone (220 mg, 62%). ¹H NMR (200 MHz, CDCl₃): δ 2.99 (s, 6H), 6.62 (dd, J=2.60 Hz, J=8.29 Hz, 1H), 7.01 (d, J=2.55 Hz, 1H), 7.08 (dt, J=1.46 Hz, J=7.18 Hz, 1H), 7.26-7.40 (m, 3H), 7.51-7.55 (m, 1H); ¹³C NMR (50 MHz, CDCl₃): δ 40.52, 108.27, 116.42, 118.86, 121.10, 124.03, 126.73, 131.88, 134.17, 134.70, 135.65, 145.95, 151.25, 194.92; Anal. Calcd for C₁₅H₁₃NO-0.4 H₂O: C, 78.17; H, 6.04. Found: C, 78.60; H, 6.00.

### EXAMPLE 12

### 3-Dimethylamino-9-fluorenone (4b)

The same reaction as described above for preparing 4a was employed, and 4b was obtained in 70% from 3-amino-9-fluorenone. ¹H NMR (200 MHz, CDCl₃): δ 3.00 (s, 6H), 6.31 (dd, J=2.31 Hz, J=8.51 Hz, 1H), 6.61 (d, J=2.28 Hz, 1H), 7.16-7.26 (m, 1H), 7.30-7.39 (m, 2H), 7.43-7.55 (m, 2H); ¹³C NMR (50 MHz, CDCl₃): δ 40.16, 102.95, 110.06, 119.41, 121.74, 122.92, 126.09, 128.68, 133.02, 136.39, 143.36, 146.72, 154.84, 191.89; Anal. Calcd for C₁₅H₁₃NO-0.2H₂O: C, 79.41; H, 5.95. Found: C, 79.59; H, 5.63.

### EXAMPLE 13

### 4-Dimethylamino-9-fluorenone (4c)

The same reaction as described above for preparing 4a was employed, and 4c was obtained in 61% from 4-amino-9-fluorenone. ¹H NMR (200 MHz, CDCl₃): δ 2.75 (s, 6H), 7.14-7.21 (m, 3H), 7.29-7.33 (m, 1H), 7.39-7.42 (m, 1H), 7.59 (d, J=7.28 Hz, 1H), 7.77 (d, J=7.52 Hz, 1H); ¹³C NMR (50 MHz, CDCl₃): δ 43.92, 118.04, 123.51, 123.98, 125.06, 127.73, 129.65, 133.65, 134.46, 135.59, 135.78, 143.98, 149.93, 193.99; Anal. Calcd for C₁₅H₁₃NO: C, 80.69; H, 5.87. Found: C, 80.66; H, 5.79.

### EXAMPLE 14

### 2-Dimethylamino-7-bromo-9-fluorenone (4d)

The same reaction as described above for preparing 4a was employed, and 4d was obtained in 18% from 2-amino-7-bromo-9-fluorenone. ¹H NMR (200 MHz, CDCl₃): δ 3.02 (s, 6H), 6.70 (dd, J=2.59 Hz, J=8.32 Hz, 1H), 7.02 (d, J=2.54 Hz, 1H), 7.16-7.32 (m, 2H), 7.48 (dd, J=1.92 Hz, J=7.94 Hz, 1H), 7.64 (d, J=1.71 Hz, 1H); ¹³C NMR (50 MHz, CDCl₃): δ 40.55, 108.42, 116.64, 120.27, 121.35, 127.30, 131.03, 135.37, 135.90, 137.07, 144.66, 151.48, 193.52; HRMS: m/z Calcd for C₁₅H₁₂BrNO: 301.0102; Found: 301.0105; Anal. Calcd for C₁₅H₁₂BrNO: C, 59.62; H, 4.00. Found: C, 59.39; H, 3.80.

### EXAMPLE 15

### 2-Dimethylamino-7-(tributylstannyl)fluorene (5)

A mixture of 2-dimethylamino-7-bromofluorene (52 mg, 0.2 mmol), bis(tributytyltin) (0.2 mL) and Pd(Ph₃P)₄ (20 mg) in a mixed solvent (12 mL, dioxane:triethylamine, 3:1) was stirred at 90° C. overnight. Solvent was removed and the residue was purified by PTLC (Hex:EtOAc, 4:1) to give 23 mg of product, 5 (yield 23%, not optimized). ¹H NMR (200 MHz, CDCl₃): δ 0.90 (t, J=7.17 Hz, 9H), 1.03-1.66 (m, 18H), 3.02 (s, 6H), 3.85 (s, 2H), 6.76 (dd, J=8.48 Hz, J=2.32 Hz, 1H), 6.94 (s, 1H), 7.37-7.64 (m, 4H); ¹³C NMR (50 MHz, CDCl₃): δ 9.67, 13.68, 27.41, 29.16, 37.06, 41.03, 109.28, 111.59, 118.20, 120.36, 131.32, 132.60, 134.54, 137.50, 141.97, 142.29, 144.83, 150.32; HRMS: m/z Calcd for C₂₇H₄₁NSn: 499.2261; Found: 499.2286.

### EXAMPLE 16

### Preparation of Radioiodinated Ligand: [125I]2f

[¹²⁵I]TZDM was prepared according to the method described previously (23). The desired [¹²⁵I]2f was prepared using iododestannylation reactions with tributyltin precursors, 12(11). Hydrogen peroxide (50 μL, 3% w/v) was added to a mixture of 50 μL of the correspondent tributyltin precursor (1μg/μL EtOH), 50 μL of 1N HCl and [¹²⁵I]NaI (1-5 mCi) in a sealed vial. The reaction was allowed to proceed for 10 min at room temperature and terminated by addition of 100 μL of sat. NaHSO₃. The reaction mixture was extracted with ethyl acetate (3×1 mL) after neutralization with saturated sodium bicarbonate solution. The combined extracts were evaporated to dryness. The residues were dissolved in 100 μL of EtOH and purified by HPLC using a reverse phase column (PRP-1, 4.6×250 mm) eluted with 100% acetonitrile—in a flow rate of 1.0 mL/min (retention time was around 12 to 13 minutes). The no-carrier-added product was evaporated to dryness and re-dissolved in 100% EtOH (1 μCi/μL). The final [¹²⁵I]2f, with a specific activity of 2,200 Ci/mmole and a greater than 95% radiochemical purity, was stored at −20° C. up to 6 weeks for autoradiography studies and animal distribution.

### EXAMPLE 17

### Dimethyl-(4′-amino-biphenyl-4-yl)-amine (2)

The mixture of dimethyl-(4′-nitro-biphenyl-4-yl)-amine (1) (1 g, 4.1 mmol) and Pd/C (200 mg, 10% pd on carbon) in a mixed solvent (150 mL, EtOAc;EtOH=2:1) was hydrogenated at 55 psi for 4 h. The mixture was filtered and the filtrate was concentrated to give clean product 2 which was used as the starting material without further purification.

¹H NMR (200 MHz, CDCl₃): 2.98 (s, 6H), 6.73 (d,t, J=8.5, 2.0 Hz, 2H), 6.80 (d,t, J=8.9, 2.0 Hz, 2H), 7.38 (d,t, J=8.5, 2.0 H, 2H), 7.44 (d,t, J=8.9,2.0 Hz, 2H). ANAL. (C₁₄H₁₆N₂)

### EXAMPLE 18

### Dimethyl-(4′-N-methylamino-biphenyl-4-yl)-amine (3)

To a mixture of 2 (100 mg, 0.47 mmol) in MeOH (10 mL) was added NaOMe solution (0.5 mL, 25% in MeOH) dropwise at RT followed by (CH₂O), (60 mg, 1.9 mmol). The resulting mixture was stirred under reflux for 2 h. NaBH4 (50 mg 1.3 mmol) was added with caution after the reaction mixture was sooled down to RT. The mixture was refluxed for 1 h and cooled down. Water (10 mL) was added followed by NaOH solution (5 mL, 1M). The mixture was extracted with CH₂CL₂. Usual work up gave crude product which was purified by PTLC (Hex:EtOAc=3:1 as developing solvent) to give 84 mg of 3 (79%).

¹H NMR (200 MHz, CDCl₃): 2.89 (s, 3H), 3.00 (s, 6H), 6.69 (d, J=8.4 Hz, 2H), 6.83(d,t, J=8.8, 2.0 Hz, 2H), 7.44 (d,t, J=8.4, 2.0 Hz, 2H), 7.49 (d,t, J=8.8,2.0 Hz, 2H). Anal. (C₁₅H₁₈N₂)

### EXAMPLE 19

### Dimethyl-(4′-N-dimethylamino-biphenyl-4-yl)-amine (4)

To a mixture of 2 (100 mg, 0.47 mmol) and (CH₂O)ₙ (200 mg, 6.3 mmol) in AcOH (5 mL) was added NaCNBH₃ (300 mg, 4.8 mmol) in one portion at RT. The mixture was stirred at RT overnight and poured into ice cold NaOH solution (15 mL, 25%). The resulting mixture was extracted with CH₂Cl₂. The organic phase was dried over Na₂SO₄, filtered, concentrated and purified by PTLC (Hex:EtOAc=3:1 as developing solvent) to give 93 mg of 4 (82%).

¹H NMR (200 MHz, CDCl₃): 2.99 (s, 12H), 6.82 (d,t, J=8.8, 2.0 Hz, 4H), 7.48 (d,t, J=8.8, 2.0 Hz, 4H). Anal. (C₁₆H₂₀N₂)

### EXAMPLE 20

### Dimethyl-(4′-hydroxy-biphenyl-4-yl)-amine (7)

A mixture of boric acid 5 (165 mg, 1 mmol) and 4-iodophenol 6 (220 mg, 1 mmol), K2CO3 (276 mg, 2 mmol) and Pd(Ph3P)4 (28 mg, 0.024 mmol) in anhydrous MeOH (5 mL) was stirred at 60° C. overnight. The mixture was filtered and washed with CH₂C₁₂. The filtrate was washed with water, dried, filtered, concentrated and purified by PTLC (Hex:EtOAc=3:1 as developing solvent) to give 125 mg of 7 (59%).

¹H NMR (200 MHz, CDCl₃): 2.98 (s, 6H), 6.80 (d,t, J=8.9, 2.0 Hz, 2H), 6.86 (d,t, J=8.7, 2.0 Hz, 2H), 7.43 (d,t, J=8.7, 2.0 Hz, 2H), 7.45 (d,t, J=8.9, 2.0 Hz, 2H). Anal. (C₁₄H₁₅NO)

### EXAMPLE 21

### Dimethyl-4-iodoaniline (9)

Same procedure described above for preparation of 4 was performed to give product 9 in 62% yield starting from 4-iodoaniline 8.

¹H NMR (200 MHz, CDCl₃): 2.92 (s, 6H), 6.49 (d,t, J=9.1, 2.0 Hz, 2H), 7.47 (d,t, J=9.1, 2.0 Hz, 2H). Anal. (C₈H₁₀IN)

### EXAMPLE 22

### Dimethyl-(3′-methoxycarbonyl-4′-amino-biphenyl-4-yl)-amine (11)

Same procedure described above for preparation of 7 was performed to give product 11 in 57% yield starting from boric acid 5 and 10.

¹H NMR (200 MHz, CDCl₃): 2.98 (s, 6H), 3.90 (s, 3H), 5.69 (br, 2H), 6.72 (d, J=8.5 Hz, 1H), 6.80 (d, J=8.8 Hz, 2H), 7.45 (d, J=8.8 Hz, 2H), 7.52 (d,d, J=8.5, 2.3 Hz, 1H), 8.08 (d, J=2.3 Hz, 1H).

### EXAMPLE 23

### Dimethyl-[3′-methoxycarbonyl-4′-(2″-p-methoxybenzylmercaptan)-acetylamino-biphenyl-4-yl]-amine (13)

To a solution of acid 12 (509 mg, 2.4 mmol) in CH₂Cl₂ (5 mL) was added a solution of oxalyl chloride (2 mL, 2 M in CH₂C₁₂) dropwise at RT followed by DMF (3 drops). The mixture was stirred at RT for 1 h. Solvent was removed on the rotavapor. To the residue was added CH₂C₁₂ (5 mL) and cold to 0° C. in an ice bath. A solution of amine 11 (541 mg, 2.0 mmol) and Et₃N (0.7 mL, 5.0 mmol) in CH₂C₁₂ (10 mL) was added dropwise at 0° C. The resulting mixture was stirred at RT for 1 h. Water was added and the organic phase was dried, filtered, concentrated and purified by flash40 (Hex:EtOAc=4:1 as eluent) to give 600 mg of 13 (65%).

¹H NMR (200 MHz, CDCl₃): 3.00 (s, 6H), 3.32 (s, 2H), 3.71 (s, 3H), 3.80 (s, 2H), 3.99 (s, 3H), 6.79 (d, J=8.7 Hz, 2H), 6.81 (d, J=8.8 Hz, 2H), 7.25 (d, J=8.7 Hz, 2H), 7.51 (d, J=8.8 Hz, 2H), 7.74 (d,d, J=8.8, 2.3 Hz, 1H), 8.23 (d, J=2.3 Hz, 1H), 8.70 (d, J=8.8 Hz, 1H).

### EXAMPLE 24

### Dimethyl-[3′-hydroxycarbonyl-4′-(2″-p-methoxybenzyhnercaptan)-acetylamino-biphenyl-4-yl]-amine (14)

A mixture of compound 13 (240 mg, 0.52 mmol) and LiOH (120 mg, 5 mmol) in mixed solvent (10 mL, THF:MeOH:H₂O=3:1:1) was stirred at RT overnight. Solvent was removed under vacuum and neutralized with 10% HCl to PH 7. The mixture was extracted with mixed solvent (CH₂Cl₂:MeOH=9:1). The organic phase was dried, filtered, concentrated to give 230 mg of acid 14 (99%) which was pure enough to run the next reaction without further purification.

¹H NMR (200 MHz, CDCl₃): 3.01 (s, 6H), 3.34 (s, 2H), 3.65 (s, 3H), 3.78 (s, 2H), 6.76 (d, J=8.4 Hz, 2H), 6.84 (d, J=8.6 Hz, 2H), 7.22 (d, J=8.5 Hz, 2H), 7.52 (d, J=8.6 Hz, 2H), 7.80 (d,d, J=8.8, 2.1 Hz, 1H), 8.33 (d, J=2.1 Hz, 1H), 8.73 (d, J=8.8 Hz, 1H).

### EXAMPLE 26

### Dimethyl-[3′-(2″-p-methoxybenzylmercaptan)-ethylaminocarbonyl-4′-(2″-p-methoxybenzylmercaptan)-acetylamino-biphenyl-4-yl]-amine (16)

To a mixture of acid 14 (230 mg, 0.51 mmol) and amine 15 (110 mg, 0.56 mmol) in CH₂Cl₂ (5 mL) was added DCC (105 mg, 0.51 mmol) in solid form followed by HOBT (69 mg, 0.51 mmol). The mixture was stirred at RT overnight. Solvent was removed after filtration and purified by flash40 (Hex:EtOAc=5:1 as eluent) to give 150 mg of 16 (47%).

¹H NMR (200 MHz, CDCl₃): 2.70 (t, J=6.4 Hz, 2H), 3.01 (s, 6H), 3.25 (s, 2H), 3.60 (q, J=6.2 Hz, 2$), 3.71 (s, 2H), 3.73 (s, 3H), 3.75 (s, 3H), 3.79 (s, 2H), 6.59 (t, J=5.4 Hz, 1H), 6.80 (d, J=8.4 Hz, 4H), 6.82 (d, J=8.6 Hz, 2H), 7.23 (d, J=8.5 Hz, 2H), 7.26 (d, J=8.5 Hz, 2H), 7.47 (d, J=8.7 Hz, 2H), 7.60 (d, J=1.9 Hz, 1H), 7.65 (d,d, J=8.5, 2.1 Hz, 1H), 8.55 (d, J=8.6 Hz, 1H), 11.42 (s, 1H).

### EXAMPLE 27

### Dimethyl-[3′-(2″-p-methoxybenzylmercaptan)-ethylaminomethyl-4′-(2″-p-methoxybenzylmercaptan)-ethylamino-biphenyl-4-yl]-amine (17)

To a solution of 16 (100 mg, 0.16 mmol) in THF (10 mL) was added BH₃-THF (3 mL, 1 M in THF) dropwise at RT. The mixture was stirred under reflux overnight. Water was added carefully to destroy the excess BH₃. Solvent was removed and to the residue was added HCl (10 mL, 10%), The mixture was refluxed for 1 h. The cold mixture was made basic with concentrated NH₄OH and extracted with mixed solvent (CH₂Cl₂:MeOH=9:1). The organic phase was dried, filtered, concentrated and purified by PTLC (CH₂Cl₂:MeOH=97:3 as developing solvent) to give 41 mg of 17 (43%).

¹H NMR (200 MHz, CDCl₃): 2.61 (q, J=6.4 Hz, 2H), 2.74 (q, J=6.7 Hz, 2H), 2.77 (q, J=6.7 Hz, 2H), 2.98 (s, 6H), 3.33 (t, J=6.7 Hz, 2H), 3.65 (s, 2H), 3.69 (s, 2H), 3.78 (s, 3H), 3.79 (s, 3H), 3.80 (s, 2H), 6.60 (d, J=8.4 Hz, 1H), 6.75-6.88 (m, 6H), 7.19-7.26 (m, 4H), 7.37 (d,d, J=8.3, 2.1 Hz, 1H), 7.45 (d, J=8.7 Hz, 3H).

### EXAMPLE 28

### Dimethyl-[3′-(2″-mercaptan)-ethylaminomethyl-4′-(2″-mercaptan)-ethylamino-biphenyl-4-yl]-amine (18)

To a solution of 17 (52 mg 0.09 mmol) in TFA (1.5 mL) was added anisole (3 drops) at RT. The mixture was cooled down to 0° C. in an ice bath. MeSO₃H (0.75 mL) was added dropwise at 0° C. The mixture was stirred at RT for 1 h. Ice water was added. The resulting mixture was extracted with ether (3 times). The aqueous phase was made basic with naHCO3 and extracted with mixed solvent (CH₂Cl₂:MeOH=9:1). The organic phase was dried, filtered, concentrated to give 26 mg of product 18 (84%).

¹H NMR (200 MHz, CDCl₃): 2.86-3.00(m, 6H), 2.97(s, 6H), 3.46-3.50 (m, 2H), 3.84 (s, 2H), 6.65-6.80 (m, 3H), 7.24 (s, 1H), 7.40-7.43 (m, 3H).

### EXAMPLE 29

### 4-amino-4-bromobiphenyl (20)

To a mixture of 4-bromo-4-nitrobiphenyl 19 (48 mg, 0.17 mmol) in 3 ml of EtOH was poured SnCl₂ (54 mg, 0.34 mmol) in one portion. The resulting mixture was stirred at reflux for 1 h, then carefully poured into saturated NaHCO₃ to make neutral solution and extracted with methylene chloride. The combine extracts were dried, filtered, and concentrated in vacuo. The residue was subjected to flash chromatography to give 30 mg of 4-amino-4-bromobiphenyl 20 (71%). ¹H NMR (200 MHz, CDCl₃): δ3.05 (s, 6H), 3.60 (br s, 2H), 6.74 (m, 2H), 7.40 (m, 4H), 7.51 (m, 2H); ¹³C NMR (50 MHz, CDCl₃): δ115.37, 120.22, 127.80, 127.92, 130.20, 131.67, 140.07, 146.16.

### EXAMPLE 30

### 4-bromo-4-dimethylaminobiphenyl (21)

To a stirred mixture of 4-amino-4-bromobiphenyl 20 (30 mg, 0.12 mmol) and papraformaldehyde (30 mg, 1 mmol) in 2 ml of AcOH at room temperature was added in one portion of NaCNBH₃. (31 mg, 0.5 mmol) The resulting mixture was stirred at room temperature for 18 h, then carefully poured into 25% aq. NaOH and ice chips to make strongly alkaline (pH 11) and extracted with methylene chloride. The combine extracts were dried, filtered, and concentrated in vacuo. The residue was subjected to flash chromatography. (EtOAc: Hex, 1:4) and gave 26 mg of 4-bromo-4-dimethylaminobiphenyl 21 (790/%). ¹H NMR (200 MHz, CDCl₃): δ3.00 (s, 6H), 6.79 (m, 2H), 7.39-7.53 (m, 6H); ¹³C NMR (50 MHz, CDCl₃): δ40.45, 112.71, 119.88, 127.47, 127.76, 131.65, 140.12, 150.16.

### EXAMPLE 31

### 4-tributylstannous-4-dimethylbiphenyl (22)

A mixture of 4-bromo-4-dimethylaminobiphenyl 21 (20 mg, 0.07 mmol), bis-(tributytyltin) (0.1 mL) and Pd(Ph₃P)₄ (10 mg) in a mixed solvent (4 mL, dioxane:triethylamine, 3:1) was stirred at 90° C. overnight. Solvent was removed and the residue was purified by PTLC (Hex:EtOAc, 4:1) to give 2.8 mg of product 22 (8%, not optinized yield). ¹H NMR (200 MHz, CDCl₃): δ0.90 (t, J=7.17 Hz, 9H), 1.6 (t, J=8.31 Hz, 6H), 1.26-1.61 (m, 12H), 2.99 (s, 6H), 6.81 (m, 2H), 7.49-7.53 (m, 61);

### EXAMPLE 32

### Iodide derivatives: 4-iodo-4-dimethylaminobiphenyl

The same procedure as that of bromide.

¹H NMR (200 MHz, CDCl₃): δ3.00 (s, 6H), 6.79 (m, 2H), 7.29 (m, 2H), 7.45 (m, 2H), 7.69 (m, 2H); ¹³C NMR (50 MHz, CDCl₃): δ40.47, 91.09, 112.70, 127.45, 127.80, 128.10, 137.64, 140.72, 150.22.

### EXAMPLE 33

### 4-Bromo-1-(4-nitrophenyl)-3-methylpyrazole (23a)

To a solution of 4-bromo-3-methylpyrazole (1.5 g, 9.31 mmol) in dry DMSO (20 mL) was added tert-BuOK (1.15 g, 10.2 mmol). After 10 min, 4-fluoronitrobenzene (1.38 g, 9.78 mmol) was added. The reaction mixture was heated to 75° C. and kept at this temperature for 1 h, it was then cooled to room temperature and quenched with water (50 mL) and extracted with ethyl acetate (50 mL). The extract was dried over Na₂SO₄ and filtered. The filtrate was concentrated and the residue was chromatographed on silica gel (1:9 ethyl acetate/hexane) to give 1.72 g of 23a (65.4 %). ¹H NMR (200 MHz, CDCl₃) δ 2.29 (s, 3H, —CH₃), 7.72 (d, J=9.2Hz, 2H, ArH), 7.91 (s, 1H, py H-5), 8.26 (d, J=9.2Hz, 2H, ArH). ¹³C NMR (200 MHz, CDCl₃) 12.56, 99.49, 118.27, 125.87, 127.78, 144.12, 145.79, 151.80. MS: m/z calcd for C₁₀H₈N₃O₂Br (M⁺) 281, found 281.Continuous elution yielded 3-bromo-1-(4-nitrophenyl)-4-methylpyrazole (23b, 128 mg, 4.8%) as a regioisomer. ¹H NMR (200 MHz, CDCl₃) δ 2.37 (s, 3H, —CH₃), 7.61 (d, J=9.1 Hz, 2H, ArH), 7.61 (s, 1H, py H-3), 8.30 (d, J=9.0 Hz, 2H, ArH).

### EXAMPLE 34

### 4-Bromo-1-(4-aminophenyl)-3-methylpyrazole (24)

A mixture of 23 (750 mg, 2.66 mmol), SnCl₂ (4.03 g, 21.3 mmol) and ethanol (20 mL) was heated at 70° C. for 2 h. After the mixture cooled to room temperature, 1 M NaOH (200 mL) was added until the mixture became alkaline. Extraction with ethyl acetate (200 mL), extraction of the combined organic layers with brine, drying over Na₂SO₄, and evaporation gave 568 mg of 24 (84.6 %). ¹H NMR (200 MHz, CDCl₃) δ 2.24 (s, 3H, —CH₃), 3.52 (br, 2H, —NH₂), 6.63 (d, J=8.7Hz, 2H, ArH), 7.27 (d, J=8.7Hz, 2H, ArH), 7.63 (s, 1H, py H-5). ¹³C NMR (200 MHz, CDCl₃) 10.96, 94.36, 114.37, 119.69, 126.28, 130.85, 144.40, 147.24. MS: m/z calcd for C₁₀H₁₁N₃Br (MH⁺) 252, found 252.

### EXAMPLE 35

### 4-Bromo-1-(4-dimethylaminophenyl)-3-methylpyrazole (25)

To a stirred mixture of 24 (500 mg, 1.98 mmol) and paraformaldehyde (594 mg, 19.8 mmol) in AcOH (20 mL) was added in one portion sodium cyanoborohydride (628 mg, 9.9 mmol) at room temperature. The resulting mixture was stirred at room temperature for 18 h, added 1 M NaOH (50 mL) and extracted with CH₂Cl₂ (50 mL×2). The organic phase was dried over Na₂SO₄ and filtered. The filtrate was concentrated and the residue was chromatographed on silica gel (1:9 ethyl acetate/hexane) gave 375 mg of 25. (67.6%). ¹H NMR (200 MHz, CDCl₃) δ 2.24 (s, 3H, —CH₃), 2.89 (s, 6H, —N(CH₃)₂), 6.65 (d, J=9.1 Hz, 2H, ArH), 7.34 (d, J=9.0 Hz, 2H, ArH), 7.63 (s, 1H, py H-5). ¹³C NMR (200 MHz, CDCl₃) 11.98, 40.63, 95.20, 112.65, 120.49, 127.22, 130.24, 148.13, 149.45. MS: m/z calcd for C₁₂H₁₄N₃Br (M⁺) 280, found 280. Anal. calcd for C₁₂H₁₄N₃Br: C 51.45, H 5.04, N 15.00, found: C, 51.65; H, 5.07; N, 14.60.

### EXAMPLE 36

### 4-(Tributylstannyl)-1-(4-dimethylaminophenyl)-3-methylpyrazole (26)

To a solution of 25 (200 mg, 0.63 mmol) in THF was added butyllithium (0.89 mL, 1.6 M in hexane) dropwise with stirring below −60° C. Then, tributyltin chloride (279 mg, 0.86 mmol) was added at such rate as to keep the reaction temperature below 0° C. The mixture was allowed to warm to room temperature and stirred overnight. After addition of water (30 mL), the mixture was extracted with ethyl acetate (30 mL×2). The extract was dried over Na₂SO₄ and filtered. The filtrate was concentrated and the residue was purified by preparative TLC (1:4 ethyl acetate/hexane) to give 124 mg of 26 (35.4%). ¹H NMR (200 MHz, CDCl₃) δ 0.83 (t, J=7.3Hz, 9H), 0.94-1.14 (m, 6H), 1.18-1.52 (m, 12H), 2.29 (s, 3H, —CH₃), 2.90 (s, 6H, N(CH₃)₂) 6.68 (d, J=9.1 Hz, 2H, ArH), 7.41 (d, J=9.1 Hz, 2H, ArH), 7.46 (s, 1H, ArH). HRMS: m/z calcd for C₂₄H₄₂N₃Sn (M) 492.2401, found 492.2423.

### EXAMPLE 37

### 4-Iodo-1-(4-dimethylaminophenyl)-3-methylpyrazole (27)

To a solution of 26 (100 mg, 0.19 mmol) in CHCl₃ (20 mL) was added a solution of iodine in CHCl₃ (0.5 mL, 1 M) at room temperature. The mixture was stirred at room temperature for 10 min. The NaHSO₃ solution (3 mL, 5% in water) and KF (3 mL, 1 M in MeOH) were added successively. The mixture was stirred for 5 min, and the organic phase was separated. The aqueous phase was extracted with CH₂Cl₂, and the combined organic phase was dried over Na₂SO₄, filtered, and concentrated to give the crude product. Preparative TLC (1:4 ethyl acetate/hexane) gave 36 mg of 27 (55.4%). ¹H NMR (200 MHz, CDCl₃) 8 2.26 (s, 3H, -CH₃), 2.90 (s, 6H, —N(CH₃)₂), 6.66 (d, J=9.1 Hz, 2H, ArH), 7.35 (d, J=9.1 Hz, 2H, ArH), 7.66 (s, 1H, py H-5). ¹³C NMR (200 MHz, CDCl₃) 12.44, 39.46, 59.76, 111.46, 119.40, 129.01, 130.58, 148.30, 150.39. MS: m/z calcd for C₁₂H₁₅N₃I (MH⁺) 328, found 328. Anal. calcd for C₁₂H₁₄N₃I: C, 44.05; H, 4.31; N, 12.84; found: C, 44.07; H, 4.24; N, 12.89.

### EXAMPLE 38

### 4-Bromo-1-(4-methylaminophenyl)-3-methylpyrazole (28)

To a mixture of 24 (100 mg, 0.40 mmol) and paraformaldehyde (52 mg, 1.99 mmol) in methanol (10 mL) was added a solution of sodium methoxide (0.5 mL, 25 wt % in methanol) dropwise at 0° C. The mixture was stirred at room temperature for 2 h. After added NaBH₄ (25 mg, 1.19 mmol), the solution was heated under reflux for 2 h. The cold mixture was added 1 M NaOH and extracted with CH₂Cl₂. The organic phase was dried over Na₂SO₄ and filtered. The filtrate was concentrated and the residue was chromatographed on silica gel (1:2 ethyl acetate/hexane) to give 45 mg of 28 (42.9 %). ¹H NMR (200 MHz, CDCl₃) δ 2.30 (s, 3H, —CH₃), 2.83 (s, 3H, —NHCH₃), 6.61 (d, J=7.2 Hz, 2H, ArH), 7.35 (d, J=7.0 Hz, 2H, ArH), 7.68 (s, 1H, py H-5). HRMS: m/z calcd for C₁₁H₁₂N₃Br (No 265.0215, found 265.0109.

### EXAMPLE 39

### 4-Bromo-1-(4-nitrophenyl)pyrazole (29)

To a solution of 4-bromopyrazole (300 mg, 2.04 mmol) in dry DMSO (10 mL) was added tert-BuOK (252 mg, 2.24 mmol). After 10 min, 4-fluoronitrobenzene (302 mg, 2.14 mmol) was added. The reaction mixture was heated to 75° C. and kept at this temperature for 2 h, it was then cooled to room temperature and quenched with water (50 mL) and extracted with ethyl acetate (50 mL). The extract was dried over Na₂SO₄ and filtered. The filtrate was concentrated to afford 420 mg of 29 (77.1%). ¹H NMR (200 MHz, CDCl₃) δ 7.68 (s, 1H, py H-3), 7.77 (d, J=9.1 Hz, 2H, ArH), 7.99 (s, 1H, py H-5), 8.29 (d, J=9.0 Hz, 2H, ArH).

### EXAMPLE 40

### 4-Bromo-1-(4-aminophenyl)pyrazole (30)

The same reaction described above to prepare 24 was employed, and 30 was obtained in 91.0% yield from 29. ¹H NMR (200 MHz, CDCl₃) δ 3.78 (br, 2H, —NH₂), 6.65 (d, J=8.8 Hz, 2H, ArH), 7.35 (d, J=8.9 Hz, 2H, ArH), 7.53 (s, 1H, py H-3), 7.70 (s, 1H, py H-5).

### EXAMPLE 41

### 4-Bromo-1-(4-dimethylaminophenyl)pyrazole (31)

The same reaction described above to prepare 25 was employed, and 31 was obtained in 81.3% yield from 30. ¹H NMR (200 MHz, CDCl₃) δ 2.83 (s, 6H, —N(CH₃)₂), 6.68 (d, J=9.1 Hz, 2H, ArH), 7.38 (d, J=9.1 Hz, 2H, ArH), 7.54 (s, 1H, py H-3), 7.72 (s, 1H, py H-5). HRMS: m/z calcd for C₁₁H₁₂N₃Br (M⁺) 265.0215, found 265.0223.

### EXAMPLE 42

### Ethyl 1-(4-nitrophenyl)-1H-pyrazole-4-carboxylate (32)

To a solution of ethyl 4-pyrazolecarboxylate (100 mg, 0.71 mmol) in dry DMSO (10 mL) was added tert-BuOK (88 mg, 0.79 mmol). After 10 min, 4-fluoronitrobenzene (106 mg, 0.75 mmol) was added. The reaction mixture was heated to 75° C. and kept at this temperature for 2 h, it was then cooled to room temperature and quenched with water (50 mL) and extracted with ethyl acetate (50 mL). The extract was dried over Na₂SO₄ and filtered. The filtrate was concentrated to afford 190 mg of crude product.

### EXAMPLE 43

### Ethyl 1-(4-aminophenyl)-1H-pyrazole-4-carboxylate (33)

The same reaction described above to prepare 24 was employed, and 33 was obtained in 84.8% yield (two step) from 32. ¹H NMR (200 MHz, CDCl₃) δ 1.30 (t, J=7.1 Hz, 3H, —CH₂CH₃), 3.16 (br, 2H, —NH₂), 4.26 (q, J=7.1 Hz, 2H, —CH₂CH₃), 6.68 (d, J=8.7 Hz, 2H, ArH), 7.38 (d, J=8.7 Hz, 2H ArH), 7.98 (s, 1H, py H-3), 8.18 (s, 1H, py H-5).

### EXAMPLE 44

### Ethyl 1-(4-dimethylaminophenyl)-1H-pyrazole-4-carboxylate (34)

The same reaction described above to prepare 25 was employed, and 34 was obtained in 41.3% yield from 33. ¹H NMR (200 MHz, CDCl₃) δ 1.29 (t, J=7.1 Hz, 3H, —CH₂CH₃), 2.92 (s, 6H, —N(CH₃)₂), 4.25 (q, J=7.1 Hz, 2H, —CH₂CH₃), 6.68 (d, J=9.1 Hz, 2H, ArH), 7.44 (d, J=9.0 Hz, 2H, ArH), 7.98 (s, 1H, py H-3), 8.19 (s, 1H, py H-5). HRMS: m/z calcd for C₁₄H₁₇N₃O₂ (M⁺) 259.1321, found 259.1320.

### EXAMPLE 45

### Binding Assays Using Aggregated Aβ40 Peptide in Solution

The solid form of peptide Aβ40 was purchased from Bachem (King of Prussia, Pa.). Aggregation of peptide was carried out by gently dissolving the peptide (0.5 mg/mL) in a buffer solution (pH 7.4) containing 10 mM sodium phosphate and 1 mM EDTA. The solutions were incubated at 37° C. for 36-42 h with gentle and constant shaking. Binding studies were carried out in 12×75 mm borosilicate glass tubes according to the procedure described (8) with some modifications. For inhibition studies, 1 mL of the reaction mixture contained 40 μl of inhibitors (concentration range between 10⁻⁵-10⁻¹⁰ M diluted in 10% EtOH), 50 μl of aggregated fibrils (10-50 nM in the final assay mixture) and 0.05 nM of radiotracer in 40% EtOH were used. The ethanol is needed for this assay, without which the some of the “cold” ligands evaluated were not soluble. Nonspecific binding was defined in the presence of 2 μM Thioflavin-T. The mixture was incubated at room temperature for 3 hr and the bound and the free radioactivity were separated by vacuum filtration through Whatman GF/B filters using a Brandel M-24R cell harvester followed by 2×3 mL washes of 10% ethanol at room temperature. Filters containing the bound I-125 ligand were counted in a gamma counter (Packard 5000) with 70% counting efficiency. The results of inhibition experiments were subjected to nonlinear regression analysis using software EBDA (15) by Kᵢ values were calculated.

Using in vitro binding assay it was demonstrated that substituted fluorenes competed with [¹²⁵I]TZDM binding to Aβ40 aggregates showing excellent binding affinities (Schemes 1-3). When fluorenes with un-methylated amino groups, 1a-1f, were tested, only 3-aminofluorene, 1b, displayed a moderate binding affinity (Kᵢ=149 nM) (Scheme 1). However, the corresponding bromo-derivative, 1d, showed a higher binding affinity (Kᵢ=56±2 nM). When the aminofluorenes were transformed to the N,N-dimethylamino derivatives (2a-2f), they dramatically increased the binding affinities to Aβ aggregates. Especially, the 7-bromo- and 7-iodo-2-N,N-dimethylaminofluorene, 2d and 2f, displayed excellent binding affinities (Kᵢ=0.85±0.1 and 0.92±0.1 nM, respectively). It is also noted that 7-N,N-dimethylamino-2-N,N-dimethylaminofluorene, 2e, also showed a very good binding affinity (Kᵢ=15.4±5 nM). The 9-hydroxyfluorenes, 3a-3d, in general showed less potency in binding to Aβ aggregates. However, 7-bromo-2-N,N-dimethylamino-9-hydroxyfluorene, 3c, displayed a moderate potency (Kᵢ=88 nM). While the corresponding 7-bromo-2-N,N-dimethylaminofluorenone, 4d, was more potent (Kᵢ=16.5±4 nM). Based on the binding data it is reasonable to conclude that for this series of fluorene derivatives with a rigid tricyclic system, a 2- or 3- substituted N,N-dimethylamino group is needed to improve binding affinity.

### EXAMPLE 46

### In Vivo Biodistribution in Normal Mice

While under ether anesthesia, 0.15 mL of a 0.1% bovine serum albumin solution containing [¹²⁵I]12f (5-10 μCi) was injected directly into the tail vein of male ICR mice (2-3 month-old, average weight 20-30 g). The mice were sacrificed by cardiac excision at various time points post injection. The organs of interest were removed and weighed, and the radioactivity was counted with an automatic gamma counter (Packard 5000). The percentage dose per organ was calculated by a comparison of the tissue counts to suitably diluted aliquots of the injected material. Total activities of blood and muscle were calculated under the assumption that they were 7% and 40% of the total body weight, respectively.

Biodistribution studies in a group of normal mice after an iv injection showed that [¹²⁵I]2f exhibited an excellent brain uptake (1.13 % ID/organ at 2 min) and peaked at 1 h (1.26% ID/organ) (Table 1). At 1 and 6 h there was 0.72 and 0.17% ID/organ, respectively remained in the brain. The blood levels are relatively low at all time point measured (4-6% 1ID/organ). The tracer seems to distribute in high blood flow areas, such as liver, kidney, muscle and skin (Table 1). The partition coefficient (P.C) of [¹²⁵I]2f is 294 (1-octanol/buffer), which is comparable to that of TZDM (P.C.=70) (23). A relatively good lipophilicity is essential for the initial brain penetration by a simple diffusion mechanism.

TABLE 1

| Organ | 2 min | | 30 min | | 60 min | | 240 min | |
|---|---|---|---|---|---|---|---|---|
| Blood | 6.50 | ±0.75 | 4.88 | ±0.90 | 4.81 | ±0.88 | 4.09 | ±0.25 |
| Heart | 1.30 | ±0.15 | 0.21 | ±0.03 | 0.19 | ±0.02 | 0.11 | ±0.02 |
| Muscle | 11.35 | ±1.43 | 9.24 | ±0.45 | 7.46 | ±1.49 | 6.21 | ±0.17 |
| Lung | 2.46 | ±0.81 | 0.76 | ±0.15 | 0.48 | ±0.06 | 0.33 | ±0.04 |
| Kidney | 4.96 | ±0.84 | 1.87 | ±0.22 | 1.46 | ±0.11 | 0.88 | ±0.10 |
| Spleen | 0.61 | ±0.08 | 0.25 | ±0.02 | 0.17 | ±0.03 | 0.18 | ±0.02 |
| Liver | 24.96 | ±2.45 | 8.84 | ±1.35 | 5.84 | ±0.88 | 5.71 | ±0.89 |
| Skin | 1.97 | ±0.68 | 7.40 | ±1.15 | 8.22 | ±0.30 | 5.66 | ±0.50 |
| Brain | 1.13 | ±0.06 | 1.26 | ±0.32 | 0.72 | ±0.03 | 0.17 | ±0.03 |

Biodistribution in mice after an intravenous injection of [¹²⁵I]2f % dose/organ, avg of 3 mice ± SD

The data in Table 2 shows the biodistribution of radiolabelled compound 27.

TABLE 2

| Organ | 2 min | | 30 min | | 1 hr | | 2 hr | |
|---|---|---|---|---|---|---|---|---|
| Blood | 6.57 | ±1.51 | 6.41 | ±0.75 | 6.09 | ±0.86 | 4.58 | ±0.13 |
| Heart | 0.82 | ±0.10 | 0.24 | ±0.07 | 0.18 | ±0.03 | 0.13 | ±0.02 |
| Muscle | 11.15 | ±0.89 | 7.87 | ±1.28 | 9.13 | ±0.51 | 5.68 | ±0.33 |
| Lung | 1.15 | ±0.18 | 0.58 | ±0.07 | 0.49 | ±0.06 | 0.43 | ±0.08 |
| Kidney | 4.39 | ±0.87 | 1.87 | ±0.60 | 1.13 | ±0.27 | 0.78 | ±0.14 |
| Spleen | 0.20 | ±0.03 | 0.14 | ±0.03 | 0.11 | ±0.01 | 0.10 | ±0.04 |
| Liver | 15.30 | ±4.50 | 4.92 | ±1.63 | 3.65 | ±1.53 | 3.01 | ±0.50 |
| Skin | 4.10 | ±0.09 | 8.75 | ±1.67 | 9.79 | ±1.12 | 7.02 | ±0.81 |
| Brain | 2.26 | ±0.36 | 0.33 | ±0.07 | 0.14 | ±0.03 | 0.09 | ±0.02 |

Biodistribution in mice after an intravenous injection of [¹²⁵I]MIPA27 % dose/organ, avg of 3 mice ± SD

### EXAMPLE 47

### In Vivo Biodistribution in Normal Mice

Tc-99m labeled compound 35 (partition coefficient of 380, obtained from third measurement, 1-Octanol/buffer) showed good brain penetration in mice. The brain uptakes were 1.18, 0.89, 0.46, 0.30% dose/organ (=2.95, 2.25, 1.15 and 0.75% dose/gram) at 2, 30 60 and 120 min. post i.v. injection, respectively.

TABLE 3

| Organ | 2 min | | 30 min | | 1 hr | | 2 hr | |
|---|---|---|---|---|---|---|---|---|
| Blood | 5.60 | ±0.79 | 2.65 | ±0.07 | 2.09 | ±0.36 | 1.52 | ±0.06 |
| Muscle | 11.31 | ±5.31 | 15.54 | ±0.83 | 15.41 | ±2.87 | 12.42 | ±0.93 |
| Lung | 2.12 | ±0.11 | 0.74 | ±0.07 | 0.69 | ±0.10 | 0.42 | ±0.05 |
| Kidney | 5.90 | ±0.67 | 2.21 | ±0.32 | 2.28 | ±0.15 | 1.70 | ±0.19 |
| Liver | 32.91 | ±2.82 | 21.65 | ±1.09 | 22.55 | ±1.00 | 17.33 | ±1.12 |
| Brain | 1.18 | ±0.20 | 0.89 | ±0.00 | 0.46 | ±0.08 | 0.30 | ±0.02 |

Biodistribution in mice after an i.v. injection of [^{99m}Tc] 35 (% dose/organ, avg. of 3 mice ± SD).

Having now fully described this invention, it will be understood to those of ordinary skill in the art that the same can be performed within a wide and equivalent range of conditions, formulations, and other parameters without affecting the scope of the invention or any embodiment thereof. All patents, patent applications, and publications cited herein are fully incorporated by reference herein in their entirety.

1. A compound of general Formula I: 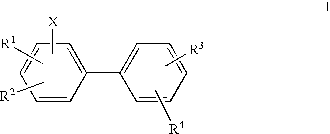
or a pharmaceutically acceptable salt thereof, wherein
R¹, R² and R³ in each instance is independently selected from the group consisting of hydrogen, halogen, C₁₋₅ alkyl, cyano, carboxy(C₁₋₅)alkyl, trifluoromethyl, nitro, methylamino, dimethylamino, halo(C₁₋₅)alkyl, hydroxy(C₁₋₅)alkyl, (Bu)₃Sn—, (Bu)₃Sn(C₁₋₅)alkyl, formyl, and the tetradentate metal ligand moeity having the following formula: 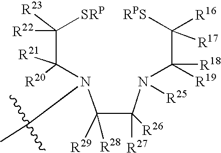
wherein,
R⁴ is selected from the group consisting of:
a. C₁₋₅ alkylthio,
b. halo(C₁₋₅)alkyl,
c. halo(C₁₋₅)alkoxy,
d. carboxy(C₁₋₅)alkyl,
e. hydroxy,
f. C₁₋₅ alkoxy,
g. hydroxy(C₁₋₅)alkyl,
h. NR⁵R⁶, wherein
R⁵ and R⁶ are independently hydrogen, halo(C₁₋₅)alkyl or C₁₋₅ alkyl,
i. phenyl(C₁₋₅)alkyl,
j. C₆₋₁₀aryl,
k. heteroaryl,
l. heterocycle,
m. heterocycle(C₁₋₅)alkyl, and
n. C₃₋₆ cycloalkyl,
wherein said phenyl(C₁₋₅)alkyl, C₆₋₁₀ aryl, heteroaryl, heterocycle, heterocycle(C₁₋₅)alkyl or C₃₋₆ cycloalkyl is substituted with one of the following: C₁₋₅ alkylthio, C₁₋₅ alkylsulfonyl, methoxy, hydroxy, dimethylamino or methylamino,
R¹⁶, R¹⁷, R¹⁸, R¹⁹, R²⁰, R²¹, R²², R²³, R²⁵, R²⁶, R²⁷, R²⁸ and R²⁹ are independently selected from the group consisting of hydrogen, halogen, C₁₋₅ alkyl, cyano, carboxy(C₁₋₅)alkyl, hydroxy(C₁₋₅)alkyl, trifluoromethyl, nitro, methylamino, dimethylamino, halo(C₁₋₅)alkyl, phenyl(C₁₋₅)alkyl, C₃₋₆ cycloalkyl, heterocycle (C₁₋₅)alkyl and carbonyl, and R^{P} is a sulfhydryl protecting group, and,
X is hydrogen, ¹²⁵I, ¹²³I, ¹³¹I, ¹⁸F, ⁷⁶Br, ⁷⁷Br or Sn(alkyl)₃.

2. A compound of claim 1, wherein
R¹, R² and R³ are hydrogen or C₁₋₅ alkyl.

3. A compound of claim 2, wherein
R¹, R² and R³ are hydrogen, and,
R⁴ is halo(C₁₋₅)alkyl, hydroxy, C₁₋₅ alkoxy or NR⁵R⁶, wherein
R⁵ and R⁶ are independently hydrogen, halo(C₁₋₅)alkyl or C₁₋₅ alkyl.

4. A compound of claim 3, wherein
R⁴ is NR⁵R⁶, wherein
R⁵ and R⁶ are independently hydrogen, halo(C₁₋₅)alkyl or C₁₋₅ alkyl.

5. A compound of claim 1, wherein
X is ¹²³I, or ¹⁸F.

6. The compound of claim 1, wherein
R¹ is methylamino or dimethylamino,
R² is hydrogen,
R³ is halo(C₁₋₅)alkyl or (Bu₃)Sn(C₁₋₅)alkyl,
R⁴ is hydroxy or hydroxy(C₁₋₅)alkyl, and,
X is hydrogen.

7. The compound of claim 6, wherein
R¹ is dimethylamino,
R³ is ¹⁸fluoro(C₁₋₅)alkyl, and,
R⁴ is hydroxy.

8. The compound of claim 7, wherein
R³ is ¹⁸fluoromethyl or ¹⁸fluoroethyl.

9. The compound of claim 8, wherein
R³ is ¹⁸fluoroethyl.

10. A compound of general Formula II: 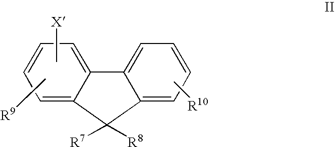
or a pharmaceutically acceptable salt thereof, wherein:
R⁹ and R¹⁰ in each instance is independently selected from the group consisting of:
a. hydrogen,
b. C₁₋₅ alkyl,
c. cyano,
d. trifluoromethyl,
e. nitro,
f. halogen,
g. hydroxy(C₁₋₅)alkyl,
h. halo(C₁₋₅)alkyl,
i. C₁₋₅ alkylthio,
j. halo(C₁₋₅)alkoxy,
k. carboxy(C₁₋₅)alkyl,
l. hydroxy,
m. C₁₋₅alkoxy,
n. NR¹¹R¹², wherein
R¹¹ and R¹² are independently hydrogen, halo(C₁₋₅)alkyl or C₁₋₅ alkyl,
o. phenyl(C₁₋₅)alkyl,
p. C₆₋₁₀ aryl,
q. heteroaryl,
r. heterocycle,
s. heterocycle(C₁₋₅)alkyl, and
t. C₃₋₆ cycloalkyl,
wherein said phenyl(C₁₋₅)alkyl, C₆₋₁₀ aryl, heteroaryl, heterocycle, heterocycle(C₁₋₅)alkyl or C₃₋₆ cycloalkyl is substituted with one of the following: C₁₋₅ alkylthio, C₁₋₅ alkylsulfonyl, methoxy, hydroxy, dimethylamino or methylamino,
u. the tetradentate metal ligand moiety having the following formula: 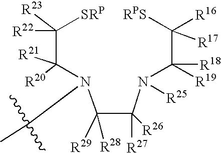
wherein, R¹⁶, R¹⁷, R¹⁸, R¹⁹, R²⁰, R²¹, R²², R²³, R²⁵, R²⁶, R²⁷, R²⁸ and R²⁹ are independently selected from the group consisting of hydrogen, halogen, C₁₋₅ alkyl, cyano, carboxy(C₁₋₅)alkyl, hydroxy(C₁₋₅)alkyl, trifluoromethyl, nitro, methylamino, dimethylamino, halo(C₁₋₅)alkyl, phenyl(C₁₋₅)alkyl, C₃₋₆ cycloalkyl, heterocycle (C₁₋₅)alkyl and carbonyl, and R^{P} is a sulfhydryl protecting group,
R⁷ and R⁸ in each instance is independently selected from the group consisting of hydrogen, hydroxy, C₁₋₅ alkyl, C₁₋₅ alkoxy, halogen, carboxy(C₁₋₅)alkyl, trifluoromethyl, and halo(C₁₋₅)alkyl, phenyl(C₁₋₅)alkyl, C₃₋₆ cycloalkyl, heterocycle(C₁₋₅)alkyl, or R⁷ and R⁸ can be taken together to form a carbonyl, and,
X′ is ¹²⁵I, ¹²³I, ¹³¹I, ¹⁸F, ⁷⁶Br, ⁷⁷Br or Sn(alkyl)₃.

11. A compound of claim 10, wherein
R⁹ is hydrogen.

12. A compound of claim 11, wherein
R⁷ and R⁸ in each instance is independently selected from the group consisting of hydrogen, hydroxyl, C₁₋₅ alkyl, halogen, and halo(C₁₋₅)alkyl, or R⁷ and R⁸ can be taken together to form a carbonyl.

13. A compound of claim 12, wherein
R¹⁰ is selected from the group consisting of cyano, nitro and NR¹¹R¹², wherein
R¹¹ and R¹² are independently hydrogen or C₁₋₅ alkyl, and,
R⁷ and R⁸ are independently hydrogen or hydroxyl.

14. A compound of claim 13, wherein
R¹⁰ is NR¹¹R¹², wherein
R¹¹ and R¹² are independently hydrogen, methyl or ethyl, and,
R⁷ and R⁸ are both hydrogen.

15. The compound of claim 14, wherein
X′ is ¹²³I or ¹⁸F.

16. A compound of general Formula III: 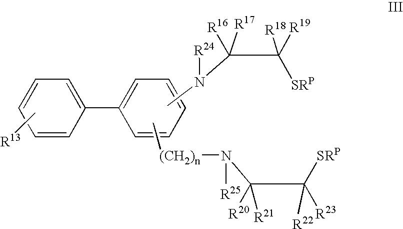
or a pharmaceutically acceptable salt thereof, wherein:
n is zero or one,
R¹³ is selected from the group consisting of:
a. C₁₋₅ alkyl,
b. cyano,
c. trifluoromethyl,
d. nitro,
e. halo(C₁₋₅)alkyl,
f. C₁₋₅ alkylthio,
g. halogen,
h. halo(C₁₋₅)alkoxy,
i. carboxy(C₁₋₅)alkyl,
j. hydroxy,
k. hydroxy(C₁₋₅)alkyl,
l. C₁₋₅ alkoxy,
m. NR¹⁴R¹⁵, wherein
R¹⁴ and R¹⁵ are independently hydrogen, halo(C₁₋₅)alkyl or C₁₋₅ alkyl,
n. phenyl(C₁₋₅)alkyl,
o. C₆₋₁₀ aryl,
p. heteroaryl,
q. heterocycle,
r. heterocycle(C₁₋₅)alkyl, and
s. C₃₋₆ cycloalkyl,
wherein said phenyl(C₁₋₅)alkyl, C₆₋₁₀ aryl, heteroaryl, heterocycle, heterocycle(C₁₋₅) cycloalkyl is substituted with one of the following: C₁₋₅ alkylthio, C₁₋₅ alkylsulfonyl, methoxy, hydroxy, dimethylamino or methylamino,
R¹⁶, R¹⁷, R¹⁸, R¹⁹, R²⁰, R²¹, R²², R²³, R²⁴ and R²⁵ in each instance is independently selected from the group consisting of hydrogen, halogen, C₁₋₅ alkyl, cyano, carboxy(C₁₋₅)alkyl, hydroxy(C₁₋₅)alkyl, trifluoromethyl, nitro, methylamino, dimethylamino, halo(C₁₋₅)alkyl, phenyl(C₁₋₅)alkyl, C₃₋₆ cycloalkyl, heterocycle, heteroaryl, C₆₋₁₀ aryl, (C₁₋₅)alkyl and carbonyl, and,
R^{P} is a sulfhydryl protecting group.

17. A compound of claim 16, wherein
R¹³ is NR¹⁴R¹⁵, wherein
R¹⁴ and R¹⁵ are independently hydrogen or C₁₋₅ alkyl.

18. A compound of claim 17, wherein
n is one,
R¹⁶ and R¹⁷ are both hydrogen or are taken together to form a carbonyl, and,
R¹⁸, R¹⁹, R²², R²³, R²⁴ and R²⁵ in each instance is independently selected from the group consisting of hydrogen and C₁₋₅ alkyl.

19. A compound of claim 18, wherein
R¹⁶, R¹⁷, R²⁰, R²¹, R²², R²³, R²⁴ and R²⁵ are hydrogen, and,
R¹⁸ and R¹⁹ are both C₁₋₅ alkyl.

20. A compound of claim 18, wherein
R¹⁶, R¹⁷, R¹⁸, R¹⁹, R²⁰, R²¹, R²⁴ and R²⁵ are hydrogen, and,
R²² and R²³ are both C₁₋₅ alkyl.

21. A compound of claim 18, wherein
R¹⁶ and R¹⁷ are taken together to form a carbonyl.

22. A compound of claim 21, wherein
R¹⁸ and R¹⁹ are both C₁₋₅ alkyl, and,
R²⁰, R²¹, R²², R²³, R²⁴ and R²⁵ are hydrogen.

23. A compound of claim 21, wherein
R¹⁸, R¹⁹, R²⁰, R²¹, R²⁴ and R²⁵ are hydrogen, and,
R²² and R²³ are both C₁₋₅ alkyl.

24. A compound of claim 21, wherein
R¹⁸, R¹⁹, R²⁰, R²¹, R²², R²³, R²⁴ and R²⁵ are hydrogen.

25. A radioisotope complex of a compound of claim 18 having the Formula: 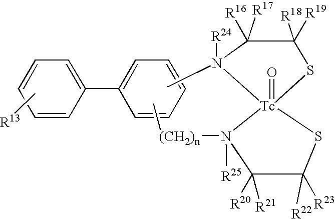
provided that one of R²⁴ and R²⁵ is selected from the group consisting of:
a. hydrogen,
b. C₁₋₅ alkyl,
c. trifluoromethyl,
d. halo(C₁₋₅)alkyl
e. carboxy(C₁₋₅)alkyl,
f. phenyl(C₁₋₅)alkyl,
g. C₆₋₁₀aryl,
h. heteroaryl
i. heterocycle,
i. heterocycle(C₁₋₅)alkyl and
k. C₃₋₆cycloalkyl
wherein said phenyl(C₁₋₅)alkyl, C₆₋₁₀ aryl, heteroaryl, heterocycle, heterocycle(C₁₋₅)alkyl or C₃₋₆ cycloalkyl is substituted with one of the following: C₁₋₅ alkylthio, C₁₋₅ alkylsulfonyl, methoxy, hydroxy, dimethylamino or methylamino,
the other of R²⁴ and R²⁵ represents an unsubstituted position.

26. A complex of claim 25, wherein
R¹³ is NR¹⁴R¹⁵, wherein
R¹⁴ and R¹⁵ are independently hydrogen or C₁₋₅ alkyl,
R¹⁶, R¹⁷, R¹⁸, R¹⁹, R²⁰ and R²¹ are hydrogen,
R²⁴ and R²⁵ are hydrogen or unsubstituted, and,
R²² and R²³ are both C₁₋₅ alkyl.

27. The complex of claim 26, wherein
R¹⁴ and R¹⁵ are independently hydrogen or methyl,
R²⁴ and R²⁵ are unsubstituted, and,
R²² and R²³ are both methyl.

28. The complex of claim 27 having the following structure:

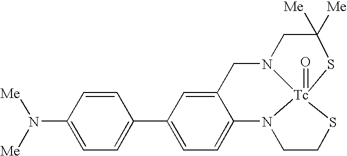

29. A compound of general Formula IV: 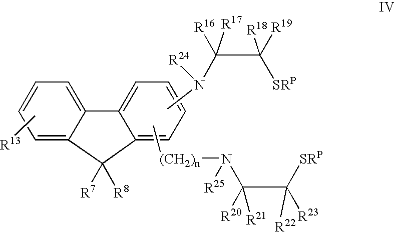
or a pharmaceutically acceptable salt thereof, wherein:
R¹³, R^{P}, R¹⁶, R¹⁷, R¹⁸, R¹⁹, R²⁰, R²¹, R²², R²³, R²⁴ and R²⁵ are as described for Formula III, and,
R⁷ and R⁸ are as described for Formula II.

30. A radioisotope complex of a compound of claim 29 having the Formula:

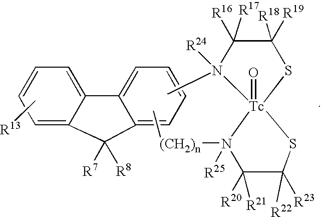

31. A compound of general Formula V: 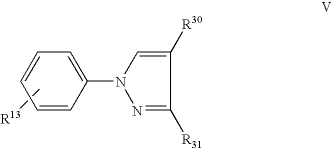
or a pharmaceutically acceptable salt thereof, wherein:
R¹³ is selected from the group consisting of:
a. C₁₋₅ alkyl,
b. cyano,
c. trifluoromethyl,
d. nitro,
e. halo(C₁₋₅)alkyl,
f. C₁₋₅ alkylthio,
g. halogen,
h. halo(C₁₋₅)alkoxy,
i. carboxy(C₁₋₅)alkyl,
j. hydroxy,
k. hydroxy(C₁₋₅)alkyl,
l. C₁₋₅ alkoxy,
m. NR¹⁴R¹⁵, wherein
R¹⁴ and R¹⁵ are independently hydrogen, halo(C₁₋₅)alkyl or C¹⁻⁵ alkyl,
n. phenyl(C₁₋₅)alkyl,
o. C₆₋₁₀ aryl,
p. heteroaryl,
q. heterocycle,
r. heterocycle(C₁₋₅)alkyl, and
s. C₃₋₆ cycloalkyl,
wherein said phenyl(C₁₋₅)alkyl, C₆₋₁₀ aryl, heteroaryl, heterocycle, heterocycle(C₁₋₅)alkyl or C₃₋₆ cycloalkyl is substituted with one of the following: C₁₋₅ alkylthio, C₁₋₅ alkylsulfonyl, methoxy, hydroxy, dimethylamino or methylamino, and,
R³⁰ and R³¹ are selected from the group consisting of hydrogen, hydroxy, hydroxy(C₁₋₅)alkyl, C₁₋₅ alkyl, C₁₋₅ alkoxy, (C₁₋₅)alkyl carboxy, halogen, carboxy(C₁₋₅)alkyl, trifluoromethyl, and halo(C₁₋₅)alkyl, phenyl(C₁₋₅)alkyl, C₃₋₆ cycloalkyl, heterocycle(C₁₋₅)alkyl, provided,
if R¹³ is other than NR¹⁴R¹⁵, wherein one of R¹⁴ and R¹⁵ is ¹⁸Fluoro(C₁₋₅)alkyl, then one of R³⁰ and R³¹ is selected from the group consisting of ¹²⁵I, ¹²³I, ¹³¹I, ¹⁸F, ⁷⁶Br, ⁷⁷Br and ¹⁸Fluoro(C₁₋₅)alkyl.

32. A compound of general Formula VI: 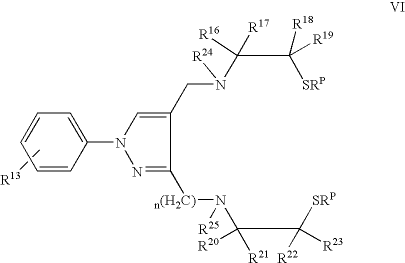
or a pharmaceutically acceptable salt thereof, wherein:
R¹³ is as described for Formula V, and,
R^{P}, R¹⁶, R¹⁷, R¹⁸, R¹⁹, R²⁰, R²¹, R²², R²³, R²⁴ and R²⁵ are as described for Formula III.

33. A radioisotope complex of a compound of claim 32 having the Formula:

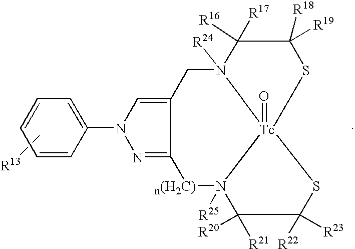

34. A pharmaceutical composition comprising a compound of any one of claims 1, 10 and 31.

35. A diagnostic composition for imaging amyloid deposits, comprising a radiolabeled compound of any one of claims 1, 10 and 31; and a pharmaceutically acceptable excipient or diluent.

36. A method of imaging amyloid deposits, comprising:
a. introducing into a mammal a detectable quantity of a diagnostic composition of claim 35; and
b. allowing sufficient time for the labeled compound to be associated with amyloid deposits; and
c. detecting the labeled compound associated with one or more amyloid deposits.
